(19) United States
(12) Patent Application Publication
(10) Pub. No.: US 2025/0190018 A1
(43) Pub. Date: Jun. 12, 2025

(54) WEARABLE DEVICE AND METHOD FOR OBTAINING DATA ABOUT USER

(71) Applicant: Samsung Electronics Co., Ltd., Suwon-si (KR)

(72) Inventors: Sungsoo CHOI, Suwon-si (KR); Sungoh KIM, Suwon-si (KR); Hyungsok YEO, Suwon-si (KR); Beomsu KIM, Suwon-si (KR); Jihyun KIM, Suwon-si (KR); Jongwon MOON, Suwon-si (KR); Donghyun YEOM, Suwon-si (KR); Valeriy PRUSHINSKIY, Suwon-si (KR)

(21) Appl. No.: 19/054,184

(22) Filed: Feb. 14, 2025

Related U.S. Application Data
(63) Continuation of application No. PCT/KR2023/009111, filed on Jun. 29, 2023.

(30) Foreign Application Priority Data
Aug. 29, 2022 (KR) 10-2022-0108769
Sep. 21, 2022 (KR) 10-2022-0119668

(51) Int. Cl. G06F 1/16; G08B 21/02

(52) U.S. Cl. CPC G06F 1/163; G08B 21/02

(57) ABSTRACT

A wearable device is provided. The wearable device includes a temperature sensor, a pupil recognition sensor, a brainwave sensor, a camera, memory storing one or more computer programs, communication circuitry, and one or more processors communicatively coupled to the temperature sensor, the pupil recognition sensor, the brainwave sensor, the camera, the communication circuitry and the memory, wherein the one or more computer programs include computer-executable instructions that, when executed by the one or more processors individually or collectively, cause the wearable device to obtain, through the temperature sensor, first data representing a temperature associated with a head of a user, contacted with the wearable device, recognize, based on the first data representing the temperature within a first reference range, a state of the user as an emergency state, based on recognizing the state of the user as the emergency state, store, in the memory, first content obtained from a second timing which is before predefined time from a first timing in which the state of the user is recognized as the emergency state through the camera, to a third timing after the predefined time from the first timing and information on a conscious state of the user obtained from the first timing to the third timing using the pupil recognition sensor or the brainwave sensor activated in response to the recognition of the emergency state, transmit, to a second wearable device contacted with a wrist, a first request to obtain second data associated with a heart rate of the user, transmit, to a first external electronic device, a second request to transmit information representing the emergency state of the user and the information on the conscious state of the user to a second external electronic device, based on the first data representing the temperature within a second reference range distinct from the first reference range, recognize the state of the user as a danger monitoring state, and based on recognizing the state of the user as the danger monitoring state, store, in the memory, second content obtained from a fourth timing in which the state of the user is recognized as the danger monitoring state through the camera, to a fifth timing after the predefined time from the fourth timing, provide information that warns of possible danger, transmit, to the second wearable device, the first request, receive, from the second wearable device, the second data in response to the first request, and identify, using the first data and the second data, whether the state of the user is changed from the danger monitoring state to the emergency state.

### CROSS-REFERENCE TO RELATED APPLICATION(S)

This application is a continuation application, claiming priority under 35 U.S.C. § 365(c), of an International application No. PCT/KR2023/009111, filed on Jun. 29, 2023, which is based on and claims the benefit of a Korean patent application number 10-2022-0108769, filed on Aug. 29, 2022, in the Korean Intellectual Property Office, and of a Korean patent application number 10-2022-0119668, filed on Sep. 21, 2022, in the Korean Intellectual Property Office, the disclosure of each of which is incorporated by reference herein in its entirety.

### BACKGROUND

### 1. Field

The disclosure relates to a wearable device and a method for obtaining data on a user.

### 2. Description of Related Art

Various services are being provided through a wearable device. The wearable device may operate by being worn on a portion of a body of a user. The wearable device may identify biometric information of the user in a state of being worn on the portion of the body of the user and provide a service based on the biometric information of the user. For example, the wearable device may identify the biometric information of the user using a plurality of sensors. The wearable device may identify various activity states of the user based on the identified biometric information of the user.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a wearable device and a method for obtaining data on a user.

Additional aspects will be set forth in part in the description which follows and, in part, will be apparent from the description, or may be learned by practice of the presented embodiments.

In accordance with an aspect of the disclosure, a wearable device is provided. The wearable device includes a temperature sensor, a pupil recognition sensor, a brainwave sensor, a camera, memory storing one or more computer programs, communication circuitry, and one or more processors communicatively coupled to the temperature sensor, the pupil recognition sensor, the brainwave sensor, the camera, the communication circuitry and the memory, wherein the one or more computer programs include computer-executable instructions that, when executed by the on ore more processors individually or collectively, cause the wearable device to obtain, through the temperature sensor, first data representing a temperature associated with a head of a user, contacted with the wearable device, recognize, based on the first data representing the temperature within a first reference range, a state of the user as an emergency state, based on recognizing the state of the user as the emergency state, store, in the memory, first content obtained from a second timing which is before predefined time from a first timing in which the state of the user is recognized as the emergency state through the camera, to a third timing after the predefined time from the first timing and information on a conscious state of the user obtained from the first timing to the third timing using the pupil recognition sensor and the brainwave sensor activated in response to the recognition of the emergency state, transmit, to a second wearable device contacted with a wrist, a first request to obtain second data associated with a heart rate of the user, and transmit, to a first external electronic device, a second request to transmit information representing the emergency state of the user and information on the conscious state of the user to a second external electronic device, based on the first data representing the temperature within a second reference range distinct from the first reference range, recognize the state of the user as a danger monitoring state, based on recognizing the state of the user as the danger monitoring state, store, in the memory, second content obtained from a fourth timing in which the state of the user is recognized as the danger monitoring state through the camera, to a fifth timing after the predefined time from the fourth timing, provide, information that warns of possible danger, transmit, to the second wearable device, the first request, receive, from the second wearable device, the second data in response to the first request, and identify, using the first data and the second data, whether the state of the user is changed from the danger monitoring state to the emergency state.

In accordance with another aspect of the disclosure, a method performed by a wearable device is provided. The method includes obtaining, by the wearable device through a temperature sensor of the wearable device, first data representing a temperature associated with a head of a user, contacted with the wearable device, recognizing, by the wearable device, based on the first data representing the temperature within a first reference range, a state of the user as an emergency state, based on recognizing the state of the user as the emergency state, storing, by the wearable device in memory of the wearable device, first content obtained from a second timing which is before predefined time from a first timing in which the state of the user is recognized as the emergency state through a camera of the wearable device, to a third timing after the predefined time from the first timing and information on a conscious state of the user obtained from the first timing to the third timing using a pupil recognition sensor of the wearable device and a brainwave sensor of the wearable device activated in response to the recognition of the emergency state, transmitting, by the wearable device to a second wearable device contacted with a wrist, a first request to obtain second data associated with a heart rate of the user, and transmitting, by the wearable device to a first external electronic device, a second request to transmit information representing the emergency state of the user and information on the conscious state of the user to a second external electronic device, based on the first data representing the temperature within a second reference range distinct from the first reference range, recognizing, by the wearable device the state of the user as a danger monitoring state, based on recognizing the state of the user as the danger monitoring state, storing, by the wearable device in the memory, second content obtained from a fourth timing in which the state of the user is recognized as the danger monitoring state through the camera, to a fifth timing after the predefined time from the fourth timing, providing, by the wearable device, information that warns of possible danger, transmitting, by the wearable device to the second wearable device, the first request, receiving, by the wearable device from the second wearable device, the second data in response to the first request, and identifying, by the wearable device, using the first data and the second data, whether the state of the user is changed from the danger monitoring state to the emergency state.

In accordance with another aspect of the disclosure, one or more non-transitory computer-readable storage media storing one or more computer programs including computer-executable instructions that, when executed by one or more processors of an wearable device individually or collectively, cause the wearable device to perform operations are provided. The operations include obtaining, by the wearable device through a temperature sensor of the wearable device, first data representing a temperature associated with a head of a user, contacted with the wearable device, recognizing, by the wearable device, based on the first data representing the temperature within a first reference range, a state of the user as an emergency state, based on recognizing the state of the user as the emergency state storing, by the wearable device in memory of the wearable device, first content obtained from a second timing which is before predefined time from a first timing in which the state of the user is recognized as the emergency state through a camera of the wearable device, to a third timing after the predefined time from the first timing and information on a conscious state of the user obtained from the first timing to the third timing using a pupil recognition sensor of the wearable device and a brainwave sensor of the wearable device activated in response to the recognition of the emergency state, transmitting, by the wearable device to a second wearable device contacted with a wrist, a first request to obtain second data associated with a heart rate of the user, transmitting, by the wearable device to a first external electronic device, a second request to transmit information representing the emergency state of the user and the information on the conscious state of the user to a second external electronic device, based on the first data representing the temperature within a second reference range distinct from the first reference range, recognizing, by the wearable device, the state of the user as a danger monitoring state, and based on recognizing the state of the user as the danger monitoring state storing, by the wearable device in the memory, second content obtained from a fourth timing in which the state of the user is recognized as the danger monitoring state through the camera, to a fifth timing after the predefined time from the fourth timing, providing, by the wearable device, information that warns of possible danger, transmitting, by the wearable device to the second wearable device, the first request, receiving, by the wearable device from the second wearable device, the second data in response to the first request, and identifying, by the wearable device, using the first data and the second data, whether the state of the user is changed from the danger monitoring state to the emergency state.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of an electronic device in a network environment according to an embodiment of the disclosure;

FIG. 2 is an example of a perspective view illustrating a wearable device according to an embodiment of the disclosure;

FIG. 3 is a simplified block diagram of a wearable device according to an embodiment of the disclosure;

FIG. 4 illustrates a specific example of a sensor of a wearable device according to an embodiment of the disclosure;

FIG. 5A illustrates an example of an environment in which a wearable device operates according to an embodiment of the disclosure;

FIG. 5B illustrates an example of an environment in which a wearable device operates according to an embodiment of the disclosure;

FIG. 6 illustrates an example of a process of a data processing in a wearable device according to an embodiment of the disclosure;

FIG. 7A illustrates an example of an operation of a wearable device according to an embodiment of the disclosure;

FIG. 7B illustrates an example of an operation of a wearable device according to an embodiment of the disclosure;

FIG. 7C illustrates an example of an operation of a wearable device according to an embodiment of the disclosure;

FIG. 7D illustrates an example of an operation of a wearable device according to an embodiment of the disclosure;

FIG. 8 is a flowchart illustrating an operation of a wearable device according to an embodiment of the disclosure;

FIG. 9A illustrates an example of an operation of a wearable device according to an embodiment of the disclosure;

FIG. 9B illustrates an example of an operation of a wearable device according to an embodiment of the disclosure;

FIG. 10 is a flowchart illustrating an operation of a wearable device according to an embodiment of the disclosure;

FIG. 11 is a flowchart illustrating an operation of a wearable device according to an embodiment of the disclosure; and

FIG. 12 is a flowchart illustrating an operation of a wearable device according to an embodiment of the disclosure.

The same reference numerals are used to represent the same elements throughout the drawings.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

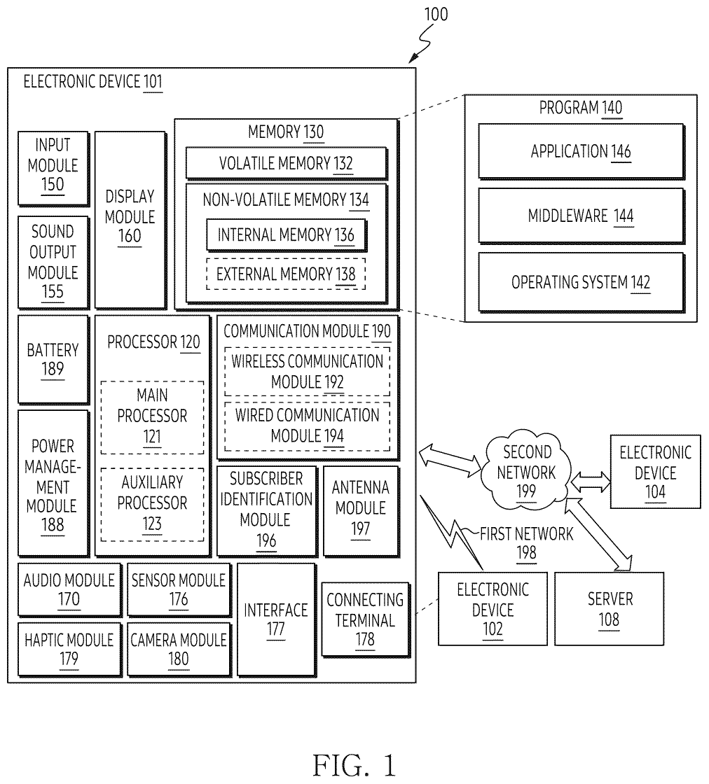

FIG. 1 is a block diagram illustrating an electronic device in a network environment according to an embodiment of the disclosure.

Referring to FIG. 1, an electronic device 101 in a network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™ wireless-fidelity (Wi-Fi) direct, or infrared data association (IrDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102 or 104, or the server 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

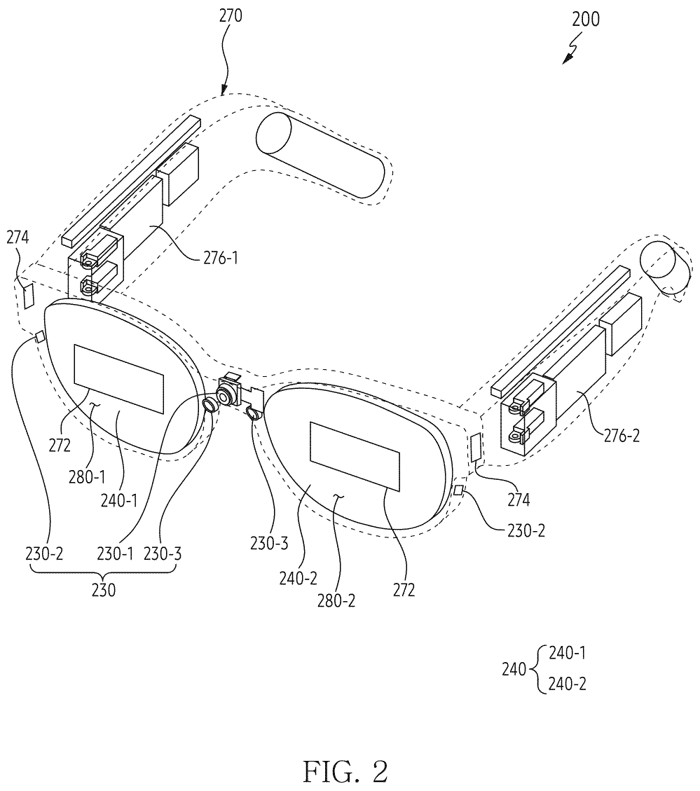

FIG. 2 is an example of a perspective view illustrating a wearable device according to an embodiment of the disclosure.

Referring to FIG. 2, a wearable device 200 may include at least a portion of components included in the electronic device 101 of FIG. 1.

In an embodiment, a frame 270 of the wearable device 200 may have a physical structure worn on a portion of a body of a user. For example, when the wearable device 200 is worn, the frame 270 may be configured so that a first display 240-1 in a display 240 is positioned in front of a right eye of the user and a second display 240-2 in the display 240 is positioned in front of a left eye of the user.

In an embodiment, the display 240 including the first display 240-1 and the second display 240-2 may include a liquid crystal display (LCD), a digital mirror device (DMD), a liquid crystal on silicon (LCoS), an organic light emitting diode (OLED), or a micro LED. In an embodiment, in case that the display 240 is configured with the LCD, the DMD, or the LCoS, the wearable device 200 may include a light source (not illustrated in FIG. 2B) that emits light toward a display area of the display 240. In an embodiment, in case that the display 240 is configured with the OLED or the micro LED, the wearable device 200 may not include the light source. However, it is not limited thereto.

In an embodiment, the wearable device 200 may further include a first transparent member 280-1 and a second transparent member 280-2. For example, each of the first transparent member 280-1 and the second transparent member 280-2 may be formed of a glass plate, a plastic plate, or a polymer. For example, each of the first transparent member 280-1 and the second transparent member 280-2 may be transparent or translucent.

In an embodiment, the wearable device 200 may include a waveguide 272. For example, the waveguide 272 may be used to transmit the light source generated by the display 240 to the eye of the user wearing the wearable device 200. For example, the waveguide 272 may be formed of glass, plastic, or the polymer. For example, the waveguide 272 may include a nano pattern configured with a lattice structure of a polygonal or a curved shape in the waveguide 272 or on a surface of the waveguide 272. For example, the light incident on an end of the waveguide 272 may be provided to the user through the nano pattern. In an embodiment, the waveguide 272 may include at least one of at least one diffractive element (e.g., a diffractive optical element (DOE), and a holographic optical element (HOE)), or a reflective element (e.g., a reflective mirror). For example, at least one diffractive element or the reflective element may be used to direct the light to the eye of the user. In an embodiment, the at least one diffractive element may include an input optical member and/or an output optical member. In an embodiment, the input optical member may mean an input grating area used as an input terminal of the light, and the output optical member may mean an output grating area used as an output terminal of the light. In an embodiment, the reflective element may include a total internal reflection optical element or a total internal reflection waveguide for total internal reflection (TIR).

In an embodiment, a camera 230 in the wearable device 200 may include a first camera 230-1, a second camera 230-2, and/or a third camera 230-3. For example, each of the first camera 230-1, the second camera 230-2, and/or the third camera 230-3 may be configured with at least one camera.

In an embodiment, the first camera 230-1 may be referred to as a high resolution (HR) or a photo video (PV) camera, and may provide an auto-focusing (AF) function or an optical image stabilization (OIS) function. In an embodiment, the first camera 230-1 may be configured with a GS camera or a remote shutter (RS) camera.

In an embodiment, the second camera 230-2 may be used for motion recognition or space recognition of three degrees of freedom (3DoF) or six degrees of freedom (6DoF). For example, the second camera 230-2 may be used for head tracking or hand detection. For example, the second camera 230-2 may be configured with a global shutter (GS) camera. For example, the second camera 230-2 may be configured with a stereo camera. For example, the second camera 230-2 may be used for gesture recognition. For example, the second camera 230-2 may identify information on a portion (e.g., a mouth) of the body of the user. As an example, the second camera 230-2 may identify information on the motion of the portion of the body of the user.

In an embodiment, the third camera 230-3 may be used to detect and track a pupil. For example, the third camera 230-3 may be configured with the GS camera. For example, the third camera 230-3 may be used to identify a user input defined by a gaze of the user.

In an embodiment, the wearable device 200 may further include an LED unit 274. For example, the LED unit 274 may be used to assist in tracking the pupil through the third camera 230-3. For example, the LED unit 274 may be configured with an IR LED. For example, the LED unit 274 may be used to compensate for brightness when illuminance around the wearable device 200 is low.

In an embodiment, the wearable device 200 may further include a first PCB 276-1 and a second PCB 276-2. For example, each of the first PCB 276-1 and the second PCB 276-2 may be used to transmit an electrical signal to a component of the wearable devices 200 such as the camera 230 or the displays 240. In an embodiment, the wearable device 200 may further include an interposer disposed between the first PCB 276-1 and the second PCB 276-2. However, it is not limited thereto.

According to an embodiment, the wearable device (e.g., the wearable device 200 of FIG. 2) may monitor a biometric signal of the user, by being worn on the user in various forms (e.g., a watch, glasses, and a band). The wearable device may obtain data on the biometric signal of the user. The wearable device may identify various states of the user based on the data on the biometric signal of the user. The wearable device may perform a designated operation or control an operation of an external electronic device according to various states of the user. In the following specification, an example of an operation of the wearable device for identifying the various states of the user and performing an operation accordingly will be described.

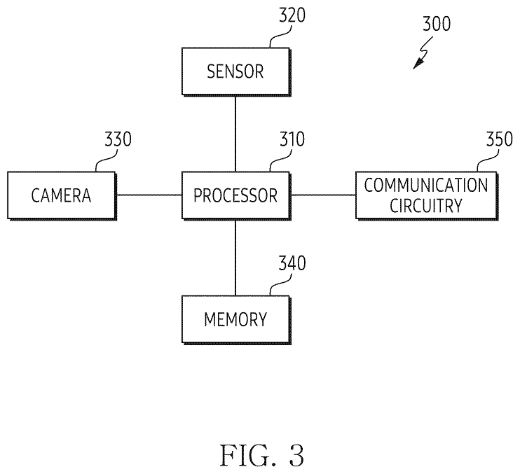

FIG. 3 is a simplified block diagram of a wearable device according to an embodiment of the disclosure.

Referring to FIG. 3, a wearable device 300 may correspond to the electronic device 101 of FIG. 1 and/or the electronic device (i.e., wearable device 200) of FIG. 2. The wearable device 300 may include a processor 310, a sensor 320, a camera 330, memory 340, and/or communication circuitry 350. In an embodiment, the wearable device 300 may include at least one of the processor 310, the sensor 320, the camera 330, the memory 340, and the communication circuitry 350. For example, at least a portion of the processor 310, the sensor 320, the camera 330, the memory 340, and the communication circuitry 350 may be omitted according to an embodiment.

According to an embodiment, the processor 310 may correspond to the processor 120 of FIG. 1. The processor 310 may be operatively (or operably) coupled with or connected with the sensor 320, the camera 330, the memory 340, and the communication circuitry 350. That the processor 310 is operatively coupled with or connected with the sensor 320, the camera 330, the memory 340, and the communication circuitry 350 may mean that the processor 310 may control the sensor 320, the camera 330, the memory 340, and the communication circuitry 350. For example, the sensor 320, the camera 330, the memory 340, and the communication circuitry 350 may be controlled by the processor 310.

According to an embodiment, the processor 310 may be configured with at least one processor. The processor 310 may include at least one processor. For example, the processor 310 may be configured with a main processor that performs high-performance processing and an auxiliary processor that performs low-power processing. At least a portion of the sensor 320 may be connected to the auxiliary processor. At least a portion of the sensor connected to the auxiliary processor may obtain data on a user for 24 hours. According to an embodiment, one of the main processor and the auxiliary processor may be activated according to a state and/or an operation of the wearable device 300. For example, in a state in which a battery of the wearable device 300 is insufficient, the auxiliary processor may be activated. For example, in a state in which accurate data on the user is required, the main processor may be activated.

According to an embodiment, the processor 310 may include a hardware component for processing data based on one or more instructions. The hardware component for processing data may include, for example, an arithmetic and logic unit (ALU), a field programmable gate array (FPGA), and/or a central processing unit (CPU).

According to an embodiment, the wearable device 300 may include the sensor 320. The sensor 320 may be used to obtain various information on the user. The sensor 320 may be used to obtain data on a body of the user. For example, the sensor 320 may be used to obtain data on a body temperature of the user, data on a motion of the user, data on a motion of a pupil, and/or data on a brainwave. For example, the sensor 320 may be configured with at least one sensor. The sensor 320 may include at least one sensor. For example, the sensor 320 may correspond to the sensor module 176 of FIG. 1.

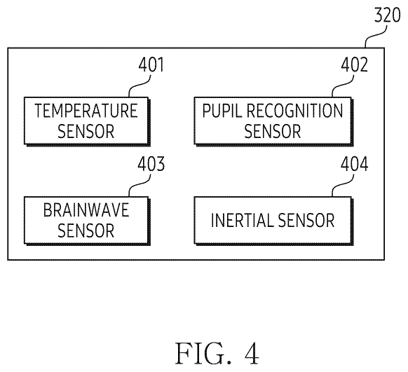

For example, the sensor 320 may include at least one of a temperature sensor (or a body temperature sensor), a pupil recognition sensor, a brainwave sensor, and an inertial sensor. A specific example of the sensor 320 including the temperature sensor, the pupil recognition sensor, the brainwave sensor, and the inertial sensor will be described later in FIG. 4.

According to an embodiment, the wearable device 300 may include the camera 330. For example, the camera 330 may include at least one camera. A first camera among the at least one camera may be used to obtain content (e.g., a video or an image) with respect to an external environment. The first camera may correspond to the first camera 230-1 illustrated in FIG. 2. A second camera among the at least one camera may be used to obtain an image with respect to a portion (e.g., a face or a hand) of the body of the user. The second camera among the at least one camera may correspond to the second camera 230-2 illustrated in FIG. 2.

According to an embodiment, the wearable device 300 may include the memory 340. The memory 340 may be used to store information or data. For example, the memory 340 may be used to store the data obtained from the user. For example, the memory 340 may correspond to the memory 130 of FIG. 1. For example, the memory 340 may be a volatile memory unit or units. For example, the memory 340 may be a non-volatile memory unit or units. For example, the memory 340 may be another type of a computer-readable medium, such as a magnetic or optical disk. For example, the memory 340 may store the data obtained based on the operation (e.g., the operation of performing an algorithm) performed in the processor 310. For example, the memory 340 may store the data obtained by the sensor 320. According to an embodiment, the memory 340 may include a buffer. The processor 310 may temporarily store the data obtained for a designated time in a buffer configured in the memory 340. The processor 310 may use the stored data in case that the data stored in the buffer is required, or delete the data stored in the buffer in case that the data is not required. For example, the data stored in the buffer may be set to be deleted after a certain period of time elapses.

The wearable device 300 may include the communication circuitry 350. The communication circuitry 350 may correspond to at least a portion of the communication module 190 of FIG. 1. For example, the communication circuitry 350 may be used for various radio access technologies (RATs). For example, the communication circuitry 350 may be used to perform Bluetooth communication or wireless local area network (WLAN) communication. For example, the communication circuitry 350 may be used to perform cellular communication. For example, the processor 310 may establish a connection with an external electronic device through the communication circuitry 350.

FIG. 4 illustrates a specific example of a sensor of a wearable device according to an embodiment of the disclosure.

Referring to FIG. 4, a sensor 320 may include a temperature sensor 401, a pupil recognition sensor 402, a brainwave sensor 403, and/or an inertial sensor 404.

For example, the temperature sensor 401 may be used to identify a temperature of a body of a user. The temperature sensor 401 may include a non-contact type infrared radiation (IR) temperature sensor or a contact-type temperature sensor. A processor 310 may measure a temperature of an object or skin in a state in which the contact-type temperature sensor is contacted with the portion (e.g., a temple) of the body of the user. The contact-type temperature sensor may include a thermocouple, a resistance temperature detector, and a thermistor. The processor 310 may measure the temperature based on infrared light through the non-contact type IR temperature sensor disposed apart from the portion of the body of the user.

For example, the pupil recognition sensor 402 may be used to obtain data on a pupil of the user. The pupil recognition sensor 402 may include a camera used to identify a gaze of the user or a pupil size and/or a sensor for iris recognition. For example, the pupil recognition sensor 402 may correspond to the third camera 230-3 of FIG. 2.

For example, the brainwave sensor 403 (or an electroencephalogram (EEG) sensor) may be used to electrically detect an electroencephalogram of the user. The brainwave sensor 403 may include at least one electrode. For example, at least one electrode of the brainwave sensor 403 may be contacted with a head (or the temple) of the user. The processor 310 may identify a potential change occurring according to an activity state of a brain of the user by using the brainwave sensor 403.

For example, the inertial sensor 404 may include an acceleration sensor and/or a gyroscope sensor. For example, the inertial sensor 404 may identify the motion of the wearable device 300 (or the user) by identifying (or measuring or detecting) the acceleration of the wearable device 300 in three directions of x-axis, y-axis, and z-axis. For example, the inertial sensor 404 may identify the motion of the wearable device 300 (or the user) by identifying (or measuring or detecting) the angular velocity of the wearable device 300 in three directions of the x-axis, y-axis, and z-axis.

The sensors (e.g., the temperature sensor 401, the pupil recognition sensor 402, the brainwave sensor 403, and the inertial sensor 404) illustrated in FIG. 4, and the sensor 320 may further include sensors for obtaining biometric data of the user. The sensor 320 may be used to identify (or detect) at least one of blood pressure, cardiogram, heart rate variability (HRV), heart rate monitor (HRM), photoplethysmography (PPG), sleep section, a skin temperature, a heart rate, blood flow, blood sugar, oxygen saturation, a pulse wave, and the electrocardiogram (ECG). For example, the processor 310 may obtain a waveform of a biometric signal based on the PPG or the ECG through the sensor 320. For example, the biometric signal may include an optical pulse wave, the pulse wave, or the electrocardiogram. The processor 310 may identify at least one of the blood pressure, the HRV, the HRM, the skin temperature, the blood flow, the blood sugar, and the oxygen saturation based on the waveform of the biometric signal.

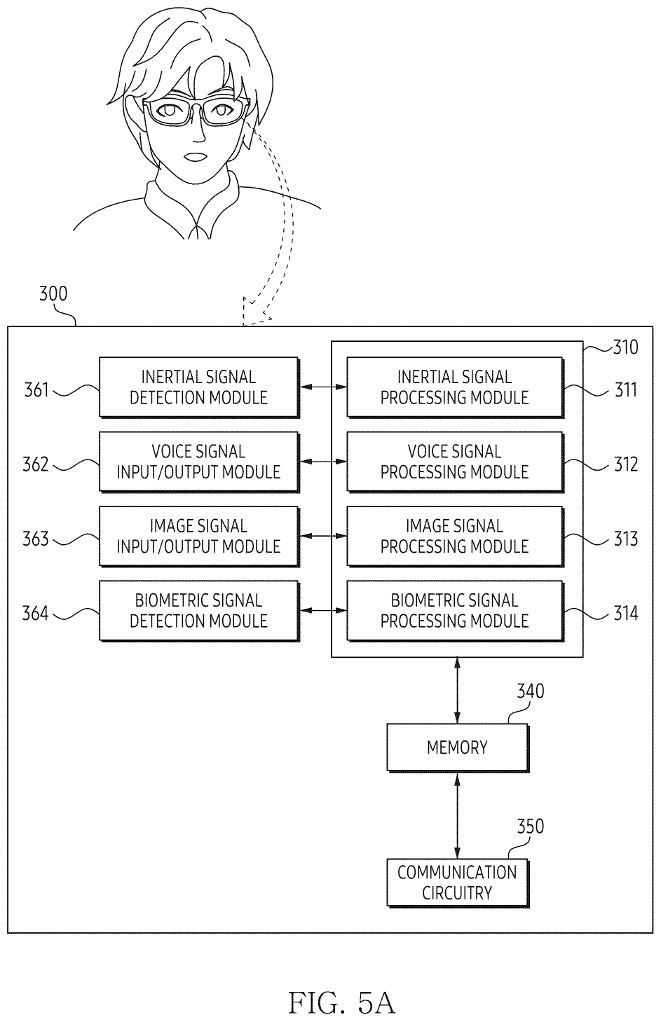

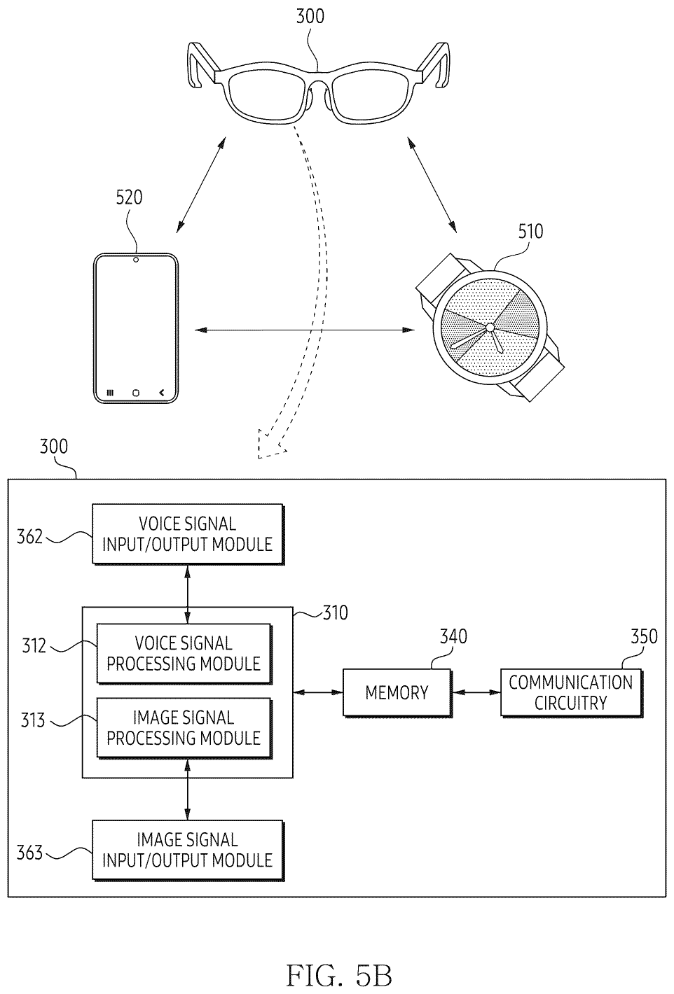

FIG. 5A illustrates an example of an environment in which a wearable device operates according to an embodiment of the disclosure.

FIG. 5B illustrates an example of an environment in which a wearable device operates according to an embodiment of the disclosure.

Referring to FIG. 5A, a wearable device 300 may operate independently without being connected (or linked) to an external electronic device (e.g., a second wearable device). For example, a processor 310 of the wearable device 300 may obtain data on a motion of the wearable device 300 (or a user) using an inertial signal detection module 361 (e.g., the inertial sensor 404). The processor 310 of the wearable device 300 may obtain data on a biometric signal of the user by using a biometric signal detection module 364 (e.g., the temperature sensor 401, the pupil recognition sensor 402, and the brainwave sensor 403). The processor 310 may store the data on the motion of the wearable device 300 and the data on the biometric signal of the user in memory 340. The processor 310 may perform a predefined operation based on the data on the motion of the wearable device 300 and the data on a motion of the user. As an example, the processor 310 may communicate with an external electronic device through a network based on the data on the motion of the wearable device 300 and the data on the motion of the user.

According to an embodiment, the wearable device 300 may include the inertial signal detection module 361, a voice signal input/output (I/O) module 362, an image signal input/output module 363, and/or the biometric signal detection module 364. For example, the inertial signal detection module 361 may include the inertial sensor 404. The voice signal input/output module 362 may include a microphone and/or a speaker. The image signal input/output module 363 may include a camera and/or a display. The biometric signal detection module 364 may include the temperature sensor 401, the pupil recognition sensor 402, the brainwave sensor 403, a photoplethysmography (PPG) sensor, and/or an electro-ocular graph (EOG) sensor.

According to an embodiment, the processor 310 may include an inertial signal processing module 311, a voice signal processing module 312, an image signal processing module 313, and/or a biometric signal processing module 314. The processor 310 may process data obtained from the inertial signal detection module 361 by using the inertial signal processing module 311. The processor 310 may process data obtained from the voice signal input/output module 362 by using the voice signal processing module 312. The processor 310 may process data obtained from the image signal input/output module 363 by using the image signal processing module 313. The processor 310 may process data obtained from the biometric signal detection module 364 by using the biometric signal processing module 314.

For example, the processor 310 may distinguish a normal state and an emergency state (e.g., a state in which a fall has occurred to the user) among states of the user, based on data on acceleration obtained through the inertial sensor 404. The processor 310 may distinguish a state in which walking of the user continues and a state in which movement is stopped after the fall, based on data on an angular velocity obtained through the inertial sensor 404.

For example, the processor 310 may obtain data on EOG, data on EEG, and/or data on PPG from the biometric signal detection module 364. The processor 310 may obtain the data on the EOG in a state in which an electrode related to an EOG sensor is contacted with skin around an eyeball of the user, using the EOG sensor. The processor 310 may obtain the data on the EEG in a state in which an electrode related to the brainwave sensor 403 is contacted with skin of forehead or the temple of the user, using the brainwave sensor 403. The processor 310 may obtain the data on the PPG, using a PPG sensor. For example, the PPG sensor may include an ear tip unit that includes a light-emitting unit to emit light of a predefined wavelength and a light-receiving unit to detect reflected light in order to obtain the data on the PPG in an ear (or an earlobe) of the user.

Referring to FIG. 5B, the wearable device 300 (e.g., a first wearable electronic device) may be connected to an external electronic device 520 and a second wearable device 510. Unlike the wearable device 300 illustrated in FIG. 5A, the wearable device 300 may operate in a state of being connected (or linked) with the external electronic device (e.g., the external electronic device 520 and the second wearable device 510).

According to an embodiment, the wearable device 300 may include the voice signal input/output (I/O) module 362 and/or the image signal input/output module 363. The voice signal input/output module 362 may include the microphone and/or the speaker. The image signal input/output module 363 may include the camera and/or the display.

According to an embodiment, the processor 310 may include the voice signal processing module 312 and/or the image signal processing module 313. The processor 310 may process the data obtained from the voice signal input/output module 362, using the voice signal processing module 312. The processor 310 may process the data obtained from the image signal input/output module 363, using the image signal processing module 313.

According to an embodiment, the second wearable device 510 may operate in a state of being worn on a portion (e.g., a hand or a wrist) of a body of the user. The second wearable device 510 may obtain data on a heart rate, data on blood pressure, and/or data on electrocardiogram. The second wearable device 510 may include the inertial sensor. The second wearable device 510 may obtain data on a motion of the second wearable device 510, using the inertial sensor included in the second wearable device 510. The second wearable device 510 may transmit at least one of the data on the heart rate, the data on the blood pressure, the data on the electrocardiogram, and the data on the motion of the second wearable device 510 to the wearable device 300.

According to an embodiment, the external electronic device 520 may be connected to a second external electronic device (e.g., a server). For example, the external electronic device 520 may be connected to the second external electronic device, using cellular communication. The external electronic device 520 may receive a request from the wearable device 300 to transmit information representing an emergency state of the user and/or information on a conscious state of the user to the second external electronic device. The external electronic device 520 may transmit the information representing the emergency state of the user and/or the information on the conscious state of the user to the second external electronic device, based on the received request.

According to an embodiment, the processor 310 of the wearable device 300 may receive various data not included in the wearable device 300 from the second wearable device 510 and/or the external electronic device 520. For example, in case that the inertial sensor 404 is not included in the wearable device 300, the data on the motion of the second wearable device 510 (or the user) may be received from the second wearable device 510 including the inertial sensor. The processor 310 may store the data received from the second wearable device 510 and/or the external electronic device 520, and identify (or recognize) the state of the user, based on the received data.

Referring to FIGS. 5A and 5B, the processor 310 may identify a trend (or a graph) for acceleration, based on the data on the acceleration of the wearable device 300 (or the user). The processor 310 may identify the trend for the acceleration greater than or equal to a predefined amplitude and configured with an irregular pattern. The processor 310 may identify that the state of the user is the emergency state (or the state in which a fall has occurred to the user), based on the trend for the acceleration that is greater than or equal to the predefined amplitude and configured with the irregular pattern. In addition, the processor 310 may identify that the state of the user is the emergency state, based on the data on the voice signal and/or the data on the image signal as well as the data on the acceleration of the wearable device 300 (or the user).

According to an embodiment, the processor 310 may identify the state of the user by configuring at least one of the data on the acceleration, the data on the voice signal, and the data on the image signal as an input value of a predefined model indicated by a plurality of parameters. For example, the predefined model may be indicated by the plurality of parameters related to a neural network. The predefined model may include a set of parameters related to the neural network. The neural network is a recognition model implemented with software or hardware that mimics computational power of a biological system, using a large number of artificial neurons (or nodes). The neural network may perform human cognitive action or a learning process through the artificial neurons. For example, parameters related to the neural network may represent a plurality of nodes included in the neural network and/or weight assigned to a connection between the plurality of nodes.

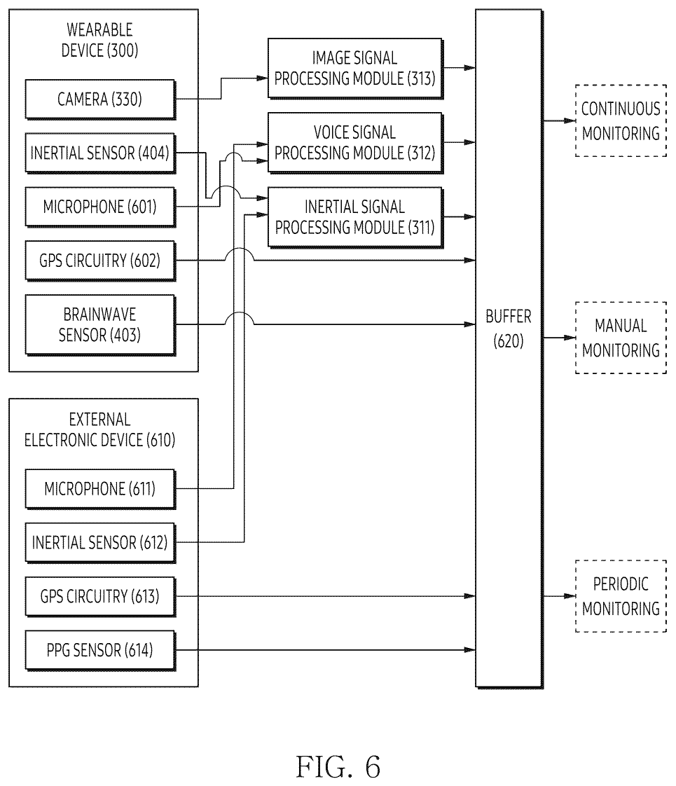

FIG. 6 illustrates an example of a process of a data processing in a wearable device according to an embodiment of the disclosure.

Referring to FIG. 6, a processor 310 may process data obtained through various components of a wearable device 300 and an external electronic device 610. A configuration of the wearable device 300 and the external electronic device 610 illustrated in FIG. 6, and the wearable device 300 and the external electronic device 610 may be configured differently according to an embodiment.

According to an embodiment, using an image signal processing module 313, the processor 310 may process data on an image signal obtained through a camera 330 included in the wearable device 300 and/or data on an image signal obtained through a camera (not illustrated) included in the external electronic device 610 (e.g., the second wearable device 510 and the external electronic device 520 of FIG. 5B). For example, the processor 310 may identify behavior of a user, using the image signal processing module 313. As an example, the processor 310 may identify that the user is walking, using the image signal processing module 313. As an example, the processor 310 may identify that the user is resting, using the image signal processing module 313. As an example, the processor 310 may identify that the user has fallen, using the image signal processing module 313. According to an embodiment, the processor 310 may store information on the identified behavior of the user, the data on the image signal obtained through the camera 330, and/or the data on the image signal obtained through the camera (not illustrated) in a buffer 620.

According to an embodiment, using a voice signal processing module 312, the processor 310 may process data on a voice signal obtained through a microphone 601 included in the wearable device 300 and/or data on a voice signal obtained through a microphone 611 included in the external electronic device 610. The processor 310 may identify a state of the voice of the user, using the voice signal processing module 312. For example, the processor 310 may identify that the voice of the user is in a normal state, using the voice signal processing module 312. For example, the processor 310 may identify that the voice of the user is in an abnormal state by using the voice signal processing module 312. According to an embodiment, the processor 310 may store information on the state of the voice of the user, the data on the voice signal obtained through the microphone 601, and the data on the voice signal obtained through the microphone 611 in the buffer 620.

According to an embodiment, using an inertial signal processing module 311, the processor 310 may process data on a motion (or an inertial signal) of the wearable device 300 (or the user) obtained through an inertial sensor 404 included in the wearable device 300 and/or data on a motion of the external electronic device 610 (or the user) obtained through an inertial sensor 612 included in the external electronic device 610. For example, the processor 310 may identify the behavior of the user, using the inertial signal processing module 311. As an example, the processor 310 may identify that the user is walking, using the inertial signal processing module 311. As an example, the processor 310 may identify that the user is resting, using the inertial signal processing module 311. As an example, the processor 310 may identify that the user has fallen, using the inertial signal processing module 311. According to an embodiment, the processor 310 may store information on the identified behavior of the user, the data on the motion of the wearable device 300 (or the user) obtained through the inertial sensor 404, and the data on the motion of the external electronic device 610 (or the user) obtained through the inertial sensor 612 in the buffer 620.

According to an embodiment, the processor 310 may store data on a location of the wearable device 300 obtained through a GPS circuitry 602 included in the wearable device 300 and/or data on a location of the external electronic device obtained through a GPS circuitry 613 included in the external electronic device 610 in the buffer 620. According to an embodiment, the processor 310 may store data on a brainwave of the user obtained through a brainwave sensor 403 included in the wearable device 300 in the buffer 620. According to an embodiment, the processor 310 may store data on a heart rate of the user obtained through a PPG sensor 614 included in the external electronic device 610 in the buffer 620.

According to an embodiment, the image signal processing module 313, the voice signal processing module 312, the inertial signal processing module 311, and the buffer 620 may be included in the processor 310 of the wearable device 300. According to an embodiment, at least a portion of the image signal processing module 313, the voice signal processing module 312, the inertial signal processing module 311, and the buffer 620 may be included in the second external electronic device (e.g., a server).

According to an embodiment, the processor 310 may delete the data stored in the buffer 620 after a predetermined time elapses. The processor 310 may delete unused data within a predetermined time.

According to an embodiment, the processor 310 may perform a continuous monitoring operation, using the data stored in the buffer 620. For example, the processor 310 may perform the continuous monitoring operation, using the data on the image signal stored in the buffer 620, the data on the voice signal stored in the buffer 620, and the data on the motion (or the inertial signal) stored in the buffer 620. According to an embodiment, the processor 310 may perform a periodic monitoring operation, using the data stored in the buffer 620. For example, the processor 310 may perform the periodic monitoring, using biometric data (e.g., the data on the brainwave and the data on the heart rate) of the user. According to an embodiment, the processor 310 may perform a manual monitoring operation, using the data stored in the buffer 620.

According to an embodiment, the processor 310 may identify the state of the user as one of a normal state, a danger monitoring state, and an emergency state by performing the monitoring operation (e.g., the continuous monitoring operation, the periodic monitoring operation, and the manual monitoring operation). For example, the normal state may mean a state in which no special problem (or event) has occurred while the user performs his or her daily life. The danger monitoring state may mean a situation in which a symptom or an event in which the danger is expected is identified, although it is not the emergency state requiring urgency from the user. The emergency state may mean a state in which an accidental risk such as a fall or cardiac arrest is identified regardless of a will of the user.

According to an embodiment, the processor 310 may train a predefined model indicated by a plurality of parameters, using the data on the motion (or the inertial signal) obtained through the inertial sensor 404. For example, the predefined model may be indicated by the plurality of parameters related to a neural network. The predefined model may include a set of parameters related to the neural network. For example, the parameters related to the neural network may represent a plurality of nodes included in the neural network and/or weight assigned to a connection between the plurality of nodes.

Reliability of the state of the user identified by the processor 310 may be improved according to the learning (or machine learning). According to an embodiment, the processor 310 may identify an event for a rapid change in the behavior of the user, using the inertial sensor 404 (e.g., an acceleration sensor and a gyroscope sensor). For example, using the inertial sensor 404, the processor 310 may identify an event in which the user stands up in the state of sitting, an event in which the user starts walking in the state of standing, and an event in which the user starts running in the state of walking. According to an embodiment, in order to identify the event for the rapid change in the behavior of the user, the processor 310 may basically use the acceleration sensor and, complementarily, use the gyroscope sensor.

According to an embodiment, the processor 310 may identify the state of user, based on the data obtained using various components (e.g., the sensor 320 and the camera 330) of the wearable device 300. For example, the processor 310 may identify that the event related to the state of the user occurs. The processor 310 may monitor a biometric signal and/or identifiable (detectable) data for the event that has occurred. The processor 310 may monitor and store a visual signal for the event that has occurred.

According to an embodiment, the processor 310 may identify that the state of the user is the normal state. While the state of the user is in the normal state, the processor 310 may identify an occurrence timing of the event related to the state of the user, using the acceleration sensor among the inertial sensor 404. In addition, the processor 310 may monitor the state of behavior of the user and the event, using the gyroscope sensor to detect a fine change in a static motion. For example, the processor 310 may identify and store (or record) the biometric signal (e.g., a body temperature, the heart rate, and blood pressure) of the user and a visual signal at the occurrence timing of the event. The processor 310 may identify that the state of the user is changed to one of the danger monitoring state and the emergency state by identifying whether the biometric signal of the user is within a first reference range or a second reference range. For example, the processor 310 may identify that the state of the user is the normal state, based on the body temperature of the user being within a normal range (e.g., greater than or equal to 35.5 degrees and less than or equal to 37.5 degrees). For example, the processor 310 may identify that the state of the user is the normal state, based on the heart rate of the user being within a normal range (e.g., greater than or equal to 50 and less than or equal to 120). For example, the processor 310 may identify that the state of the user is the normal state, based on the blood pressure of the user being within a normal range (e.g., greater than or equal to 80 [mmHg] and less than or equal to 120 [mmHg]).

According to an embodiment, the processor 310 may identify that the state of the user is the danger monitoring state. The processor 310 may identify that the state of the user is the danger monitoring state, based on identifying whether the biometric signal of the user is within the second reference range. For example, the processor 310 may identify that the state of the user is the danger monitoring state, based on identifying that the temperature of the user is out of the normal range. For example, the processor 310 may identify that the state of the user is the danger monitoring state, based on identifying that the heart rate of the user is out of the normal range. For example, the processor 310 may identify that the state of the user is the danger monitoring state, based on identifying that the blood pressure of the user is out of the normal range. The processor 310 may identify and store (or record) the biometric signal (e.g., the body temperature, the heart rate, and the blood pressure) of the user and the visual signal from a second timing which is before predefined time from a first timing in which the biometric signal of the user is identified within the second reference range, to a third timing after the predefined time from the timing.

According to an embodiment, the processor 310 may identify that an external object is approaching the user, based on the visual signal. The processor 310 may identify information on a size and velocity of the external object. The processor 310 may identify that the external object approaches the user at a high speed. The processor 310 may identify possible danger to the user. According to an embodiment, the processor 310 may identify that noise greater than reference noise level is generated, based on the voice signal. The processor 310 may identify the possible danger to the user, based on the voice signal.

According to an embodiment, the processor 310 may identify that the state of the user is the emergency state. For example, in the danger monitoring state, the processor 310 may identify the state of the user as the emergency state, based on that the biometric signal is changed from the second reference range to the first reference range, or that a number of times or time changed to the first reference range satisfies a designated condition. For example, the processor 310 may identify the state of the user as the emergency state based on identifying that a fall or impact has occurred to the user.

For example, the processor 310 may identify the state of the user as the emergency state, based on identifying the occurrence of a motion (e.g., a fall) of the user greater than or equal to the size of a reference movement and/or the noise greater than or equal to a reference level, based on the visual signal and/or the voice signal.

For example, the processor 310 may identify and store the data on the event related to the emergency state, based on the state of the user being the emergency state. The processor 310 may transmit information representing that the user is in the emergency state to the external electronic device. The processor 310 may provide notification for notifying that the user is in the emergency state.

According to an embodiment, the first reference range and second reference range for identifying the described state of the user may be set, based on a gender, an age, and a body mass index (BMI) of the user. In case that the size of data obtained for the designated time after using the wearable device 300 is appropriate to perform the machine learning, the processor 310 may set (or update) the first reference range and the second reference range, using the machine learning, based on the data for the user.

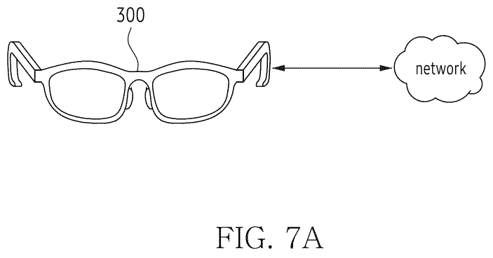

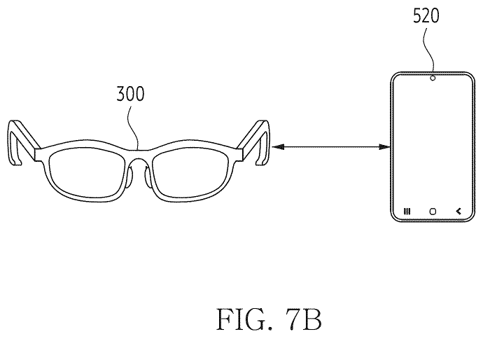

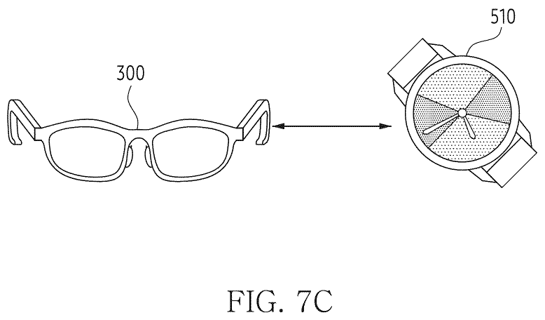

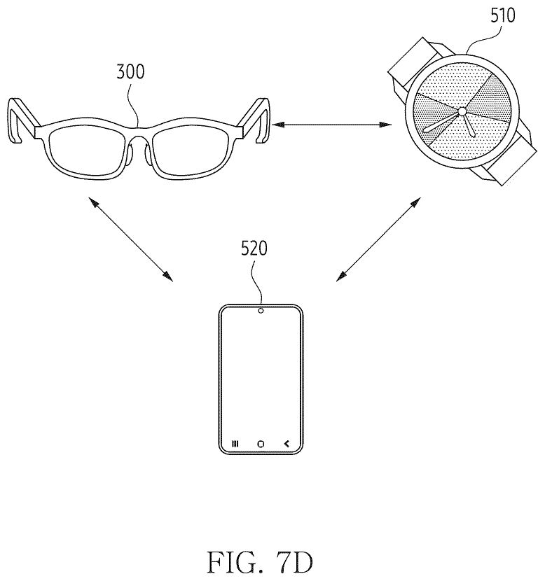

FIG. 7A illustrates an example of an operation of a wearable device according to an embodiment of the disclosure.

FIG. 7B illustrates an example of an operation of a wearable device according to an embodiment of the disclosure.

FIG. 7C illustrates an example of an operation of a wearable device according to an embodiment of the disclosure.

FIG. 7D illustrates an example of an operation of a wearable device according to an embodiment of the disclosure.

Referring to FIG. 7A, a wearable device 300 may operate independently without being connected to an external electronic device. For example, the wearable device 300 may be connected to the external electronic device (e.g., a server) through a network.

According to an embodiment, the wearable device 300 may include a temperature sensor 401, a camera 330, a pupil recognition sensor 402, an inertial sensor 404, and/or a PPG sensor. For example, at least one electrode related to the temperature sensor 401 may be disposed in a frame of the wearable device 300. For example, an ear tip unit of the PPG sensor may be contact with an ear (or an earlobe) of a user.

According to an embodiment, the processor 310 may identify data on a motion (or an inertial signal) of the user by using the inertial sensor 404 (e.g., an acceleration sensor and a gyroscope sensor) while a state of the user is a normal state. The processor 310 may identify an event for a change in behavior of the user. The processor 310 may monitor a biometric signal (e.g., a body temperature, a PPG, and a heart rate) of a user at an occurrence timing of the identified event. The processor 310 may identify the state of the user as an emergency state, based on identifying that the biometric signal (e.g., the body temperature, the PPG, and the heart rate) of the user being within a first reference range. For example, the processor 310 may identify the state of the user as the emergency state, based on that a number of times or time that the biometric signal of the user is changed to the first reference range satisfies a designated condition. For example, the processor 310 may identify that a fall has occurred to the user, based on the data on the motion (or the inertial signal) obtained through the inertial sensor 404. The processor 310 may identify the state of the user as the emergency state, based on identifying that the fall has occurred to the user.

According to an embodiment, the processor 310 may identify the state of the user as the emergency state. For example, while the state of the user is the emergency state, the processor 310 may identify a first timing in which an event that caused the change to the emergency state occurred. The processor 310 may identify and store data on a visual signal, using the camera 330 from a second timing which is before predefined time from the first timing to a third timing after the predefined time from the first timing. For example, the processor 310 may provide information representing a warning for an emergency situation. The processor 310 may transmit the information representing the warning for the emergency situation to the external electronic device (e.g., a server), using communication circuitry 350. The processor 310 may provide the user with the information representing the warning for the emergency situation.

According to an embodiment, the processor 310 may identify the state of the user as a danger monitoring state, based on identifying that the biometric signal of the user is changed within the second reference range. While the state of the user is a danger expected state, the processor 310 may identify a fourth timing in which an event that caused the change to the danger expected state occurred. The processor 310 may identify and store the data on the visual signal, using the camera 330 from a fifth timing which is before predefined time from the fourth timing to a sixth timing after the predefined time from the first timing. According to an embodiment, the processor 310 may identify and store the data on the visual signal, using the camera 330 from the fourth timing to the sixth timing.

According to an embodiment, the processor 310 may identify whether the state of the user is changed from the danger monitoring state to the emergency state, while the state of the user is the danger expected state. The processor 310 may identify whether the state of the user is changed from the danger monitoring state to the emergency state, by monitoring the heart rate and/or heart rate variability of the user, using the PPG sensor. The processor 310 may identify whether the state of the user is changed from the danger monitoring state to the emergency state by performing a pupil response test and/or a pupil contraction/relaxation test, using the pupil recognition sensor 402.

According to an embodiment, the processor 310 may use components included in the wearable device 300 to identify (or recognize) the state of the user. The processor 310 may change the detection period of the signal (e.g., the biometric signal, the inertial signal, or the visual signal) according to the state of the user. For example, the processor 310 may identify and store data (e.g., data on the biometric signal, the data on the motion (or the inertial signal), and the data on the visual signal) with respect to the user identified before and after the event that caused the change to the danger expected state. For example, the processor 310 may identify and store the data (e.g., the data on the biometric signal, the data on the motion (or the inertial signal), and the data on the visual signal) with respect to the user identified before and after the event that caused the change to the emergency state.

According to an embodiment, an example of an operation of the components of the wearable device 300 according to the state of the user may be set as illustrated in Table 1.

TABLE 1

| signal | | | | danger | |
|---|---|---|---|---|---|
| detection | detection | detection | normal | monitoring | |
| module | device | information | state | state | emergency state |
| inertial | acceleration | normal | state of | state of | state of a fall |
| signal | sensor | state | sitting | walking | |
| detection | | posture | state of | state of | |
| module | | change in | standing | running | |
| | | stationary | | state of | |
| | | state | | jumping | |
| | gyroscope | posture | — | rapid | rapid change in |
| | sensor | change in | | change in | behavior |
| | | moving | | behavior | |
| | | state | | | |
| voice | microphone | user voice | — | — | — |
| signal | | ambient | — | — | — |
| input/output | | noise | | | |
| module | | heart sound | — | — | — |
| | speaker | — | | | |
| image | pupil | left and | pupil | pupil | pupil |
| signal | recognition | right pupil | contraction | contraction/ | contraction/ |
| input/output | sensor, | image | (sympathetic | expansion | expansion |
| module | camera | | nerve | identification | identification |
| | | | activity)/ | comparison | comparison of |
| | | | expansion | of left | left and right |
| | | | (parasympathetic | and right | pupil size |
| | | | activity) | pupil size | |
| | | | identification | | |
| | | pupil | — | identifying | identifying no |
| | | response | | that the | change in pupil |
| | | information | | rate of | size due to light |
| | | | | change in | |
| | | | | pupil size | |
| | | | | due to | |
| | | | | light is | |
| | | | | reduced | |
| biometric | PPG sensor | heart rate | identifying | identifying | identifying |
| signal | | | within | deviation | deviation from |
| detection | | | normal | from | normal range |
| module | | | range (60- | normal | and atrial |
| | | | 100) | range | fibrillation |
| | temperature | body | identifying | identifying | identifying |
| | sensor | temperature | within | deviation | deviation from |
| | | | normal | from | normal range |
| | | | range | normal | |
| | | | (36.5 | range | |
| | | | degrees to | | |
| | | | 37.5 | | |
| | | | degrees) | | |
| | EOG sensor | eye | — | — | — |
| | | movement | | | |

Referring to FIG. 7B, the wearable device 300 may operate in a state of being connected to an external electronic device 520. For example, the external electronic device 520 may include the inertial sensor for identifying the motion of the user. The processor 310 of the wearable device 300 may receive data on the motion of the user from the external electronic device 520. The processor 310 may identify the state of the user, based on the data on the motion of the user. According to an embodiment, the processor 310 may identify the state of the user as the normal state. The processor 310 may monitor the biometric signal of the user while the state of the user is the normal state. The processor 310 may identify the data on the motion of the user, using the external electronic device 520 while the state of the user is the normal state. The processor 310 may identify an event for the change in the behavior of the user, by receiving the data on the motion of the user. According to an embodiment, the processor 310 may receive information on a timing in which the event for the change in the behavior of user occurred from the external electronic device 520.

According to an embodiment, the processor 310 may identify the state of the user as the emergency state. For example, an operation of the wearable device 300 while the state of the user is the emergency state may correspond to the operation of the wearable device 300 of FIG. 7A.

According to an embodiment, the processor 310 may identify the state of the user as the danger monitoring state. For example, an operation of the wearable device 300 while the state of the user is the danger monitoring state may correspond to the operation of the wearable device 300 of FIG. 7A.

According to an embodiment, the wearable device 300 may identify the data on the motion of the user by using the inertial sensor included in the external electronic device 520. The processor 310 may reduce power consumption of the wearable device 300 using the external electronic device 520 to identify the data on the motion of the user.

According to an embodiment, the processor 310 of the wearable device 300 may identify the state of the user by controlling an operation of components included in the external electronic device 520. For example, in case that a component included in the wearable device 300 is also included in the external electronic device 520, the processor 310 may identify data on the user, using at least one of the component included in the wearable device 300 and the component included in the external electronic device 520. As an example, in case that both the component included in the wearable device 300 and the component included in the external electronic device 520 are used, the processor 310 may obtain more accurate data by combining (or comparing) the data obtained from the external electronic device 520 and the wearable device 300, respectively.

According to an embodiment, the external electronic device 520 may include a large-capacity battery and various sensors compared to the wearable device 300. Therefore, in case that the wearable device 300 operates in the state of being connected (or linked) to the external electronic device 520, the power consumption of the wearable device 300 may be reduced and the state of the user may be identified. For example, the external electronic device 520 may operate in a state that the inertial sensor and an illuminance sensor are always turned on. Therefore, in case that the user wears the wearable device 300 while carrying the external electronic device 520, the external electronic device 520 may detect the motion of the user in real time. The processor 310 may activate the camera 330 of the wearable device 300 and identify (or monitor) and store (or record) data on the visual signal before and after a designated event only when the designated event occurs based on the motion of the user.

For example, an example of the operation of the components of the external electronic device 520 according to the state of the user may be set as illustrated in Table 2.

TABLE 2

| signal | | | | danger | |
|---|---|---|---|---|---|
| detection | detection | detection | normal | monitoring | emergency |
| module | device | information | state | state | state |
| inertial | acceleration | normal | state of | state of | state of a fall |
| signal | sensor | state | sitting | walking | |
| detection | | posture | state of | state of | |
| module | | change in | standing | running | |
| | | stationary | | state of | |
| | | state | | jumping | |
| | gyroscope | posture | — | rapid change | rapid change |
| | sensor | change in | | in behavior | in behavior |
| | | moving | | | |
| | | state | | | |
| voice | microphone | user voice | — | — | — |
| signal | | ambient | — | — | — |
| input/output | | noise | | | |
| module | speaker | — | | | |
| image | camera | surrounding | obtaining | obtaining | obtaining |
| signal | | image | user image | user image | user image |
| input/output | | information | and | and | and |
| module | | | surrounding | surrounding | surrounding |
| | | | image | image | image |
| | illuminance | ambient | — | identifying | identifying |
| | sensor | illuminance | | rapid | rapid |
| | | | | illuminance | illuminance |
| | | | | change | change |
| biometric | PPG sensor | heart rate | identifying | identifying | identifying |
| signal | | | within | deviation | deviation |
| detection | | | normal | from normal | from normal |
| module | | | range (60- | range | range and |
| | | | 100) | | atrial |
| | | | | | fibrillation |
| | temperature | body | identifying | identifying | identifying |
| | sensor | temperature | within | deviation | deviation |
| | | | normal | from normal | from normal |
| | | | range | range | range |
| | | | (36.5 | | |
| | | | degrees to | | |
| | | | 37.5 | | |
| | | | degrees) | | |
| | ECG sensor | heart | — | ECG | — |
| | | activity | | measurement | |
| | | | | and recording | |

Referring to FIG. 7C, the wearable device 300 may operate in a state of being connected to a second wearable device 510. For example, the second wearable device 510 may include various components for identifying the biometric signal (e.g., the PPG, the heart rate, the body temperature, and/or the blood pressure). The processor 310 of the wearable device 300 may receive data on the biometric signal from the second wearable device 510. The processor 310 may identify the state of the user, based on the data on the biometric signal. According to an embodiment, the processor 310 may identify the state of the user as the normal state. The wearable device 300 and the second wearable device 510 may identify data on the motion of the user, using the inertial sensor, respectively. The processor 310 of the wearable device 300 may receive the data on the motion of the user from the second wearable device 510. The processor 310 may identify an event for a change in the behavior of the user, based on the data on the motion of the user identified in the wearable device 300 and the data on the motion of the user received from the second wearable device 510.

According to an embodiment, the processor 310 may identify the state of the user as the emergency state. For example, while the state of the user is the emergency state, the processor 310 may identify a first timing in which an event that caused a change to the emergency state occurred. The processor 310 may identify and store data on the visual signal, using the camera 330 from a second timing which is before predefined time from the first timing to a third timing after the predefined time from the first timing. For example, the processor 310 may provide information representing a warning for the emergency situation. The processor 310 may transmit the information representing the warning for the emergency situation to the external electronic device (e.g., the server), using the communication circuitry 350. The processor 310 may provide the user with the information representing the warning for the emergency situation.

For example, while the state of the user is the emergency state, the second wearable device 510 may identify the data on the biometric signal, using a sensor (e.g., the temperature sensor and the PPG sensor) to identify the biometric signal of the user from the second timing to the third timing. The second wearable device 510 may transmit the data on the biometric signal to the wearable device 300.

According to an embodiment, the processor 310 may identify the state of the user as the danger monitoring state, based on identifying that the biometric signal of the user is changed within the second reference range. While the state of the user is the danger expected state, the processor 310 may identify a fourth timing in which an event that caused a change to the danger expected state occurred. The processor 310 may identify and store the data on the visual signal, using the camera 330 from a fifth timing which is before predefined time from the fourth timing to a sixth timing after the predefined time from the first timing.

For example, while the state of the user is the emergency state, the second wearable device 510 may identify the data on the biometric signal, using a sensor (e.g., the temperature sensor and the PPG sensor) to identify the biometric signal of the user from the fifth timing to the sixth timing. The second wearable device 510 may transmit the data on the biometric signal to the wearable device 300.

According to an embodiment, the wearable device 300 may be worn on a first portion (e.g., a head) of a body of the user. The second wearable device 510 may be worn on a second portion (e.g., a wrist) of the body of the user. For example, data on the motion (or the inertial signal) may be obtained from both the wearable device 300 and the second wearable device 510. In order to increase efficiency of a computational operation, the processor 310 of the wearable device 300 may set to obtain the data on the motion (or the inertial signal) and the data on the biometric signal in the second wearable device 510. The processor 310 may obtain data on a time signal while the data on the motion (or the inertial signal) and the data on the biometric signal are obtained in the second wearable device 510. The processor 310 may reduce the power consumption by setting the second wearable device 510 to obtain the data on the motion (or the inertial signal) and the data on the biometric signal.

For example, an example of the operation of the components of the second wearable device 510 according to the state of the user may be set as illustrated in Table 3.

TABLE 3

| signal | | | | danger | |
|---|---|---|---|---|---|
| detection | detection | detection | normal | monitoring | emergency |
| module | device | information | state | state | state |
| inertial | acceleration | normal | state of | state of | state of a fall |
| signal | sensor | state | sitting | walking | |
| | | posture | state of | state of | |
| | | change in | standing | running | |
| | | stationary | | state of | |
| | | state | | jumping | |
| detection | gyroscope | posture | — | rapid change | rapid change |
| module | sensor | change in | | in behavior | in behavior |
| | | moving | | | |
| | | state | | | |
| voice | microphone | user voice | — | — | — |
| signal | | ambient | — | — | — |
| input/output | | noise | | | |
| module | speaker | — | | | |
| biometric | PPG sensor | heart rate | identifying | identifying | identifying |
| signal | | | within | deviation | deviation |
| detection | | | normal | from normal | from normal |
| module | | | range (60- | range | range and |
| | | | 100) | | atrial |
| | | | | | fibrillation |
| | temperature | body | identifying | identifying | identifying |
| | sensor | temperature | within | deviation | deviation |
| | | | normal | from normal | from normal |
| | | | range | range | range |
| | | | (36.5 | | |
| | | | degrees to | | |
| | | | 37.5 | | |
| | | | degrees) | | |
| | ECG sensor | heart | — | ECG | — |
| | | activity | | measurement | |
| | | | | and recording | |

Referring to FIG. 7D, the wearable device 300 may operate in a state of being connected to the second wearable device 510 and the external electronic device 520. The second wearable device 510 may include various components for identifying the biometric signal (e.g., the PPG, the heart rate, the body temperature, and/or the blood pressure). The processor 310 of the wearable device 300 may receive the data on the biometric signal from the second wearable device 510. The external electronic device 520 may include the inertial sensor for identifying the motion of the user. The processor 310 of the wearable device 300 may receive the data on the motion of the user from the external electronic device 520. The processor 310 may identify the state of the user, based on the data on the biometric signal received from the second wearable device 510 and the data on the motion of the user received from the external electronic device 520.

According to an embodiment, as the processor 310 set the second wearable device 510 and the external electronic device 520 to perform a portion of the operation performed in the wearable device 300 illustrated in FIG. 7A, the data monitoring operation for the user may be efficiently performed.

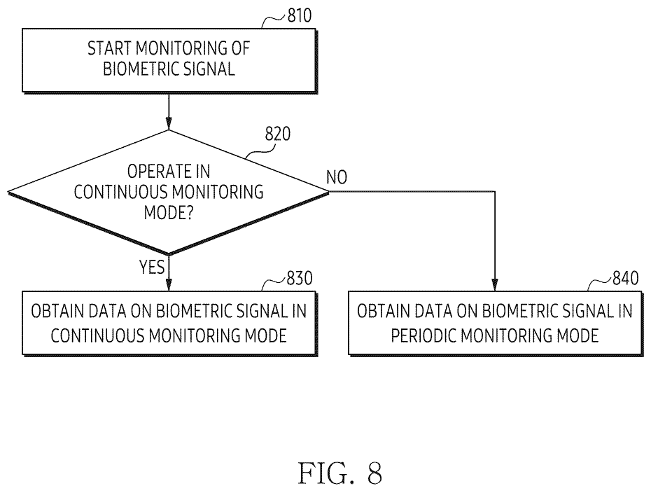

FIG. 8 is a flowchart illustrating an operation of a wearable device according to an embodiment of the disclosure.

Referring to FIG. 8, in an operation 810, a processor 310 may start monitoring a biometric signal of a user, using a sensor 320.

According to an embodiment, the processor 310 may start monitoring the biometric signal of the user in response to a user input. The processor 310 may receive the user input indicating to monitor the biometric signal of the user. The processor 310 may start monitoring the biometric signal of the user in response to the received user input.

According to an embodiment, the processor 310 may start monitoring the biometric signal of the user, based on identifying that a designated event occurs.

In an operation 820, the processor 310 may identify whether a wearable device 300 operates in a continuous monitoring mode. For example, the wearable device 300 may operate in one of the continuous monitoring mode and a periodic monitoring mode.

For example, by operating in the continuous monitoring mode, the processor 310 may continuously monitor the biometric signal for the elderly and/or a patient with severe cardiovascular disease whose condition may change suddenly into an emergency state.

For example, by operating in the periodic monitoring mode, the processor 310 may periodically monitor the biometric signal (e.g., a body temperature, blood pressure, and heart rate variability) for a user with chronic adult disease (e.g., diabetes or high blood pressure).

In an operation 830, in case that the wearable device 300 operates in the continuous monitoring mode, the processor 310 may obtain the data on the biometric signal of the user according to the continuous monitoring mode. For example, the processor 310 may continuously (or for 24 hours) identify whether the biometric signal of the user is within a normal range.

In an operation 840, in case that the wearable device 300 operates in the periodic monitoring mode, the processor 310 may obtain the data on the biometric signal of the user according to the periodic monitoring mode. The processor 310 may obtain the data on the biometric signal, based on a designated time interval (e.g., 24 hours).

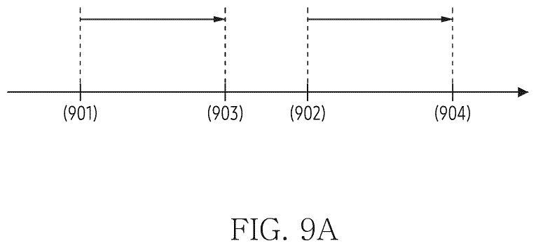

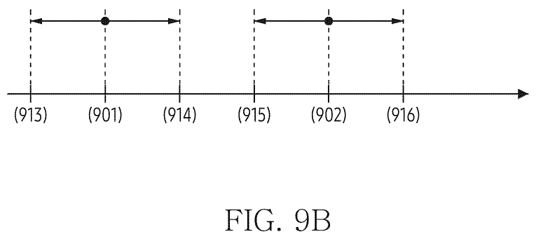

FIG. 9A illustrates an example of an operation of a wearable device according to an embodiment of the disclosure.

FIG. 9B illustrates an example of an operation of a wearable device according to an embodiment of the disclosure.

Referring to FIG. 9A, a processor 310 (or a wearable device 300) may operate the wearable device 300 in one of a manual monitoring mode for obtaining data on a user in response to a user input and an automatic monitoring mode for obtaining data on the user based on satisfying a predefined condition.

For example, the manual monitoring mode and the automatic monitoring mode may be classified according to whether the user input is received. For example, a continuous monitoring mode and a periodic monitoring mode may be classified according to a period of obtaining (or measuring) data on the user.

According to an embodiment, the processor 310 (or the wearable device 300) may operate in the manual monitoring mode. The processor 310 may operate in one of the continuous monitoring mode and the periodic monitoring mode, based on the user input. The processor 310 may obtain the data on the user by operating in one of the continuous monitoring mode and the periodic monitoring mode, based on the user input. The processor 310 may reduce power consumption by obtaining the data (e.g., data on a biometric signal, data on a motion (or an inertial signal)) on the user, based on the user input.

For example, at timing 901, the processor 310 may receive a first user input for obtaining the data on the user from the user who feels an abnormal symptom of his or her condition. The processor 310 may recognize, based on the first user input, a state of the user as a danger monitoring state. The processor 310 may obtain and store (or record), based on the first user input, data on the biometric signal (e.g., electrocardiogram, blood pressure, and a body temperature). For example, the first user input may be set as one of an input to a button included in a housing of the wearable device 300, an input through voice recognition, and a touch input to a display of the wearable device 300.

For example, the processor 310 may obtain the data on the biometric signal of the user from the timing 901 to timing 903 after predefined time from the timing 901. According to an embodiment, the processor 310 may obtain the data on the biometric signal of the user from the timing 901 until another user input distinguished from the first user input is received.

According to an embodiment, the processor 310 may identify that the state of the user is changed from a normal state or the danger monitoring state to an emergency state. For example, the processor 310 may recognize the state of the user as the emergency state, based on identifying that a fall has occurred to the user. According to an embodiment, the processor 310 may receive a second user input at timing 902 after recognizing the state of the user as the emergency state. The processor 310 may identify and store the data on the biometric signal of the user and data on a visual signal from the timing 902 to timing 904 after predefined time, in response to the second user input received at the timing 902. For example, the processor 310 may identify and store information on a conscious state of the user from the timing 902 to the timing 904, using a pupil recognition sensor 402 and a brainwave sensor 403 activated in response to identifying that the state of the user is changed to the emergency state, based on the second user input.

According to an embodiment, the processor 310 (or the wearable device 300) may operate in the automatic monitoring mode. The processor 310 may operate in one of the continuous monitoring mode and the periodic monitoring mode, based on satisfying the predefined condition. The processor 310 may obtain the data on the user, by operating in one of the continuous monitoring mode and the periodic monitoring mode, based on satisfying the predefined condition. For example, the processor 310 may obtain the data on the user, based on that behavior or the state of the user satisfies the predefined condition. For example, the processor 310 may operate in the automatic monitoring mode in case that continuously monitoring the abnormal symptom of the user is required.

For example, at the timing 901, the processor 310 may identify that the behavior or the state of user satisfies the predefined condition. The processor 310 may obtain and store (or record) the data on the biometric signal (e.g., the electrocardiogram, the blood pressure, and the body temperature), based on identifying that the behavior or the state of user satisfies the predefined condition. According to an embodiment, the processor 310 may obtain the data on the biometric signal of the user from the timing 901 to the timing 903 after the predefined time from the timing 901. According to an embodiment, the processor 310 may obtain the data on the biometric signal of the user from the timing 901 until another predefined condition is satisfied.

For example, at the timing 902, the processor 310 may identify that the state of the user is changed from the normal state or the danger monitoring state to the emergency state. In response to identifying that the state of the user is changed to the emergency state, the processor 310 may identify and store the data on the biometric signal of the user and the data on the visual signal from the timing 902 to the timing 904 after the predefined time. For example, the processor 310 may identify and store information on the conscious state of the user from the timing 902 to the timing 904, using the pupil recognition sensor 402 and the brainwave sensor 403 activated in response to identifying that the state of the user is changed to the emergency state.

Referring to FIG. 9B, the processor 310 (or the wearable device 300) may operate in the manual monitoring mode. For example, at the timing 901, the processor 310 may receive the first user input for obtaining the data on the user from the user who feels the abnormal symptom of his or her condition. The processor 310 may recognize, based on the first user input, the state of the user as the danger monitoring state. The processor 310 may obtain and store (or record), based on the first user input, the data on the biometric signal (e.g., the electrocardiogram, the blood pressure, and the body temperature).

For example, the processor 310 may obtain the data on the biometric signal of the user from timing 913 which is before predefined time from the timing 901 to timing 914 after the predefined time from the timing 901. For example, the processor 310 may obtain the data on the biometric signal from the timing 913 to the timing 901, and store the obtained data on the biometric signal in a buffer configured in memory 340. The processor 310 may obtain the data on the biometric signal of the user from the timing 901 to the timing 914, based on receiving the first user input at the timing 901. By combining the data on the biometric signal from the timing 913 to the timing 901 stored in the buffer and the data on the biometric signal of the user from the timing 901 to the timing 914, the processor 310 may obtain the data on the biometric signal of the user from the timing 913 to the timing 914.

For example, the processor 310 may identify that the state of the user is changed from the normal state or the danger monitoring state to the emergency state. The processor 310 may receive the second user input at the timing 902 after recognizing the state of the user as the emergency state. The processor 310 may identify and store the data on the biometric signal of the user and the data on the visual signal from timing 915 which is before the predefined time from the timing 902 to timing 916 after the predefined time from the timing 902, in response to the second user input received at the timing 902. For example, the processor 310 may identify and store, based on the second user input, information on the conscious state of the user from the timing 915 to the timing 916, using the pupil recognition sensor 402 and the brainwave sensor 403 activated in response to identifying that the state of the user is changed to the emergency state.

According to an embodiment, the processor 310 (or the wearable device 300) may operate in the automatic monitoring mode. The processor 310 may operate in one of the continuous monitoring mode and the periodic monitoring mode, based on satisfying the predefined condition. For example, the processor 310 may obtain the data on the user, based on the behavior or the state of the user satisfying the predefined condition.

For example, at the timing 901, the processor 310 may identify that the behavior or the state of the user satisfies the predefined condition. The processor 310 may obtain the data on the biometric signal from the timing 913 which is before the predefined time from the timing 901 to timing 914 after the predefined time from the timing 901, based on identifying that the behavior or the state of the user satisfies the predefined condition. For example, the processor 310 may obtain the data on the biometric signal from the timing 913 to the timing 901, and store the obtained data on the biometric signal in the buffer configured in the memory 340. The processor 310 may obtain the data on the biometric signal of the user from the timing 901 to the timing 914, based on that behavior or the state of the user satisfies the predefined condition. By combining the data on the biometric signal obtained from the timing 913 to the timing 901 stored in the buffer and the data on the biometric signal of the user from the timing 901 to the timing 914, the processor 310 may obtain the data on the biometric signal of the user obtained from the timing 913 to the timing 914.

For example, the processor 310 may identify that the state of the user is changed from the normal state or the danger monitoring state to the emergency state. At the timing 902, the processor 310 may identify that the behavior or the state of the user satisfies another predefined condition. The processor 310 may recognize the state of the user as the emergency state, in response to identifying that the behavior or the state of the user satisfies another predefined condition at the timing 902.

For example, based on identifying that the behavior or the state of the user satisfies the predefined condition, the processor 310 may identify and store the data on the biometric signal of the user and the data on the visual signal from the timing 915 which is before the predefined time from the timing 902 to the timing 916 after the predefined time from the timing 902. For example, the processor 310 may identify and store, based on identifying that the behavior or the state of the user satisfies the predefined condition, the information on the conscious state of the user from the timing 915 to the timing 916, using the pupil recognition sensor 402 and the brainwave sensor 403 activated in response to identifying that the state of the user is changed to the emergency state.

Referring to FIGS. 9A and 9B, the processor 310 may set data on the user to be obtained while the state of the user is the emergency state. For example, the processor 310 may set to obtain data on the visual signal and the data on a brainwave signal while the state of the user is the emergency state.

According to an embodiment, the processor 310 may determine data on the user obtained according to the state of the user, based on components included in the wearable device 300. For example, in case that the wearable device 300 is set to operate independently without being connected (or linked) to an external electronic device, the processor 310 may obtain the data on the motion (or the inertial signal) and the data on the biometric signal of the user.

For example, in case that the wearable device 300 is set to operate in a state of being connected to a second wearable device 510 and an external electronic device 520, the processor 310 may control the external electronic device 520 to obtain the data on the motion (or the inertial signal), using an inertial sensor included in the external electronic device 520. The processor 310 may control the second wearable device 510 to obtain the data on the biometric signal, using a sensor for detecting the biometric signal included in the second wearable device 510. The processor 310 may obtain the data on the visual signal by using a camera 330 while the data on the motion (or the inertial signal) and the data on the biometric signal are obtained.

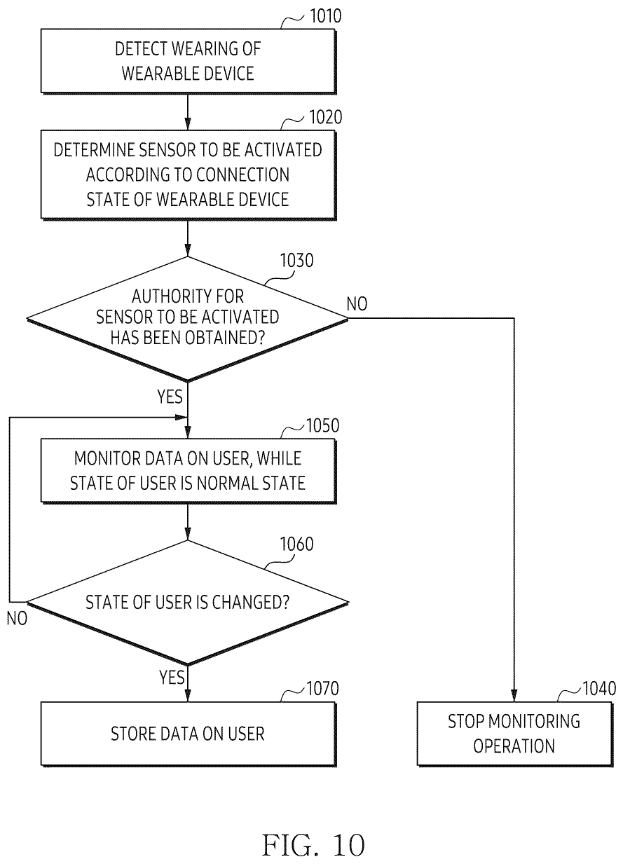

FIG. 10 is a flowchart illustrating an operation of a wearable device according to an embodiment of the disclosure.

Referring to FIG. 10, in an operation 1010, a processor 310 may detect wearing of a wearable device 300. For example, the processor 310 may detect the wearing of the wearable device 300, based on identifying a change in impedance of an electrode (e.g., an electrode related to an ECG sensor) included in the wearable device 300. For example, the processor 310 may detect the wearing of the wearable device 300, based on identifying a pupil of a user, using a pupil recognition sensor 402.

In an operation 1020, the processor 310 may determine sensors to be activated according to a connection state of the wearable device 300. The processor 310 may identify an external electronic device (e.g., a second wearable device 510 and an external electronic device 520) connected to the wearable device 300. The processor 310 may identify sensors included in the external electronic device connected to the wearable device 300. The processor 310 may determine the sensors to be activated based on identifying the sensors included in the external electronic device. For example, the processor 310 may operate by being worn on a wrist of the user and may identify the external electronic device including a PPG sensor. The processor 310 may cause the external electronic device to obtain data on a heart rate of the user, using the PPG sensor included in the external electronic device. For example, in case that at least one sensor included in the wearable device 300 is also included in the external electronic device, the processor 310 may deactivate the at least one sensor of the wearable device 300 and activate the at least one sensor included in the external electronic device.

According to an embodiment, the processor 310 may monitor a state of the user, using sensors (e.g., a pupil recognition sensor 402) having low power consumption in the wearable device 300. The processor 310 may additionally activate sensors (e.g., a brainwave sensor 403) having high power consumption, based on that the state of the user satisfies a designated condition. For example, the processor 310 may additionally activate the sensors (e.g., the brainwave sensor 403) having high power consumption, based on recognizing the state of the user as a danger monitoring state and/or an emergency state. For example, the processor 310 may identify whether an abnormal pattern occurs or whether the obtained data is out of a threshold range, based on the data obtained by using the sensors having low power consumption. As an example, the processor 310 may additionally activate the sensors having high power consumption, based on identifying that an abnormal pattern occurs in the obtained data. As an example, the processor 310 may additionally activate the sensors having high power consumption, based on identifying that the obtained data is out of the threshold range.

In an operation 1030, the processor 310 may identify whether authority for sensors to be activated has been obtained. The processor 310 may identify whether the authority has been obtained not only for sensors included in the wearable device 300 but also for sensors included in the external electronic device connected to the wearable device 300. For example, the processor 310 may request authority from the user for the sensor to be activated, using the wearable device 300 or the external electronic device connected to the wearable device 300.

In an operation 1040, in case that the authority for the sensors to be activated is not obtained, the processor 310 may stop a monitoring operation. For example, the processor 310 may stop the monitoring operation for obtaining data on the user, based on identifying that the authority for the sensors to be activated is not obtained. For example, the processor 310 may refrain from the monitoring operation for obtaining the data on the user.

In an operation 1050, in case that the authority for the sensors to be activated is obtained, the processor 310 may monitor the data on the user, while the state of the user is a normal state. For example, the processor 310 may monitor the data on the user, based on identifying that the authority has been obtained for the sensors to be activated. For example, the processor 310 may obtain data on a motion (or an inertial signal), using an inertial sensor included in the wearable device 300 or the external electronic device connected to the wearable device 300. The processor 310 may obtain the data on the motion of the user by obtaining the data on the motion (or the inertial signal).

In an operation 1060, the processor 310 may identify whether the state of the user is changed. The processor 310 may identify whether the state of the user is changed, while monitoring the data on the user. According to an embodiment, in case that the state of the user is not changed, the processor 310 may perform the operation 1050 again.

For example, the processor 310 may identify whether the state of the user is changed from the normal state to the danger monitoring state. For example, the processor 310 may identify whether the state of the user is changed from the normal state to the emergency state.

In an operation 1070, in case that the state of the user is changed, the processor 310 may store (or record) the data on the user. For example, based on identifying that the state of the user is changed, the processor 310 may store the data on the user.

According to an embodiment, the processor 310 may store the data on the user, based on identifying that the state of the user is changed from the normal state to the danger monitoring state or the emergency state. For example, the processor 310 may identify a timing when the state of the user is changed from the normal state to the danger monitoring state or the emergency state. The processor 310 may identify the data on the user before and after the identified timing. For example, the processor 310 may store content (e.g., a video, an image, a voice, a call record (or substance), position information of the wearable device 300, and information on an application being executed) obtained before and after the identified timing. As an example, the processor 310 may store data on a biometric signal of the user obtained before and after the identified timing.

According to an embodiment, the processor 310 may provide notification to the user representing whether to share the data on the user before and after the identified timing to a designated contact or an emergency institution. After the notification is provided, the processor 310 may share the data on the user to the designated contact or the emergency institution, or may store the data on the user in memory 340 according to a user input.

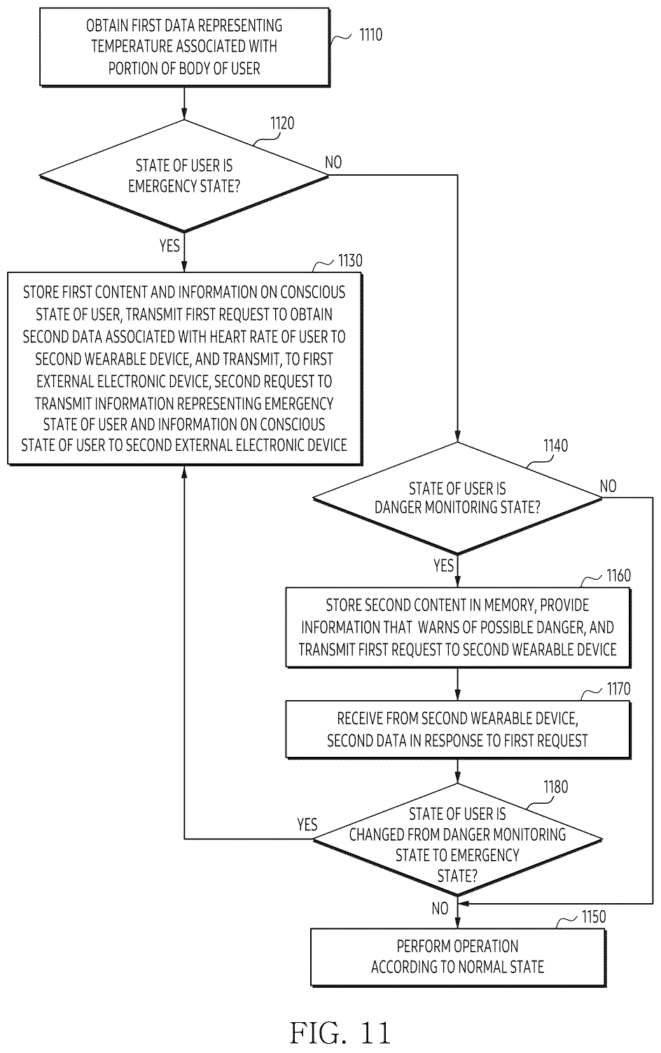

FIG. 11 is a flowchart illustrating an operation of a wearable device according to an embodiment of the disclosure.

Referring to FIG. 11, in an operation 1110, a processor 310 may obtain first data representing a temperature associated with a portion (e.g., a head) of a body of a user. For example, the processor 310 may obtain, through a temperature sensor 401, the first data representing the temperature associated with the head of the user, contacted with a wearable device 300.

According to an embodiment, the wearable device 300 may be worn on the head of the user. A portion (e.g., an electrode) of the wearable device 300 may be contacted with the head of the user. Based on identifying that the wearable device 300 is worn by the user, the processor 310 may obtain, through the temperature sensor 401, the first data representing the temperature associated with the head of the user.

According to an embodiment, the processor 310 may obtain, based on a first time interval, the first data. For example, the first time interval may be set by the user.

In an operation 1120, the processor 310 may identify whether a state of the user is an emergency state. For example, the processor 310 may identify, based on the first data representing the temperature within a first reference range, whether the state of the user is the emergency state.

For example, the processor 310 may identify a first value representing a number of times that the temperature is identified within the first reference range, the temperature being obtained based on the first time interval in predefined time resource. The processor 310 may identify whether the state of the user is the emergency state, according to whether the first value is greater than or equal to a predefined value.

In an operation 1130, based on identifying (or recognizing) the state of the user as the emergency state, the processor 310 may store first content (e.g., a video, an image, a voice, a call record (or content), position information of the wearable device 300, and information on an application being executed) and information on a conscious state of the user, transmit a first request to obtain second data associated with a heart rate of the user to a second wearable device, and transmit, to a first external electronic device, a second request to transmit information representing the emergency state of the user and the information on the conscious state of the user to a second external electronic device (e.g., a server).

For example, based on identifying that the first value representing a number of times that is identified within the first reference range is greater than or equal to the predefined value, the processor 310 may identify that the state of the user is the emergency state.

For example, the processor 310 may identify the first content (e.g., the video, the image, the voice, the call record (or content), the position information of the wearable device 300, and the information on the application being executed) obtained from a second timing which is before predefined time from a first timing in which the state of the user is identified (or recognized) as the emergency state, to a third timing after the predefined time from the first timing. The processor 310 may obtain the first content obtained, using a camera 330, from the second timing to the third timing. The processor 310 may store the obtained first content in memory 340. For example, the processor 310 may store, in a buffer configured in the memory 340, third content (e.g., the video, the image, the voice, the call record (or content), the position information of the wearable device 300, and the information on the application being executed) obtained using the camera 330 from the second timing to the first timing. The processor 310 may obtain fourth content (e.g., the video, the image, the voice, the call record (or content), the position information of the wearable device 300, and the information on the application being executed) from the first timing to the third timing, in response to recognizing the state of the user as the emergency state. The processor 310 may obtain the first content by combining the third content stored in the buffer and the fourth content. For example, the third content may be a first video of a surrounding environment of the user obtained from the second timing to the first timing. The fourth content may be a second video of a surrounding environment of the user obtained from the first timing to the third timing. The processor 310 may obtain a third video by splicing the first video and the second video with respect to the first timing. The processor 310 may obtain the first content by obtaining the third video from the second timing to the third timing.

For example, the processor 310 may activate a pupil recognition sensor 402 or a brainwave sensor 403 in response to identification (or recognition) of the emergency state. The processor 310 may obtain information on the conscious state of the user obtained from the first timing in which the state of the user is identified (or recognized) as the emergency state to the third timing after the predefined time from the first timing, using the activated pupil recognition sensor 402 and the brainwave sensor 403. The processor 310 may store, in the memory 340, the information on the conscious state of the user obtained from the first timing to the third timing.

For example, the processor 310 may transmit, to the second wearable device contacted with a wrist of the user, the first request to obtain the second data associated with the heart rate of the user. The processor 310 may identify that the second wearable device includes a PPG sensor. The processor 310 may control for obtaining the second data on the heart rate of the user by transmitting the first request to the second wearable device.

For example, the processor 310 may transmit, to the first external electronic device, the second request to transmit the information representing the emergency state of the user and the information on the conscious state of the user to the second external electronic device. For example, the first external electronic device and the wearable device 300 may be connected based on a first radio access technology (RAT) (e.g., Bluetooth communication). The first external electronic device may be connected to the second external electronic device based on a second RAT (e.g., cellular communication). The processor 310 may transmit the second request to the first external electronic device to transmit the information representing the emergency state of the user and the information on the conscious state of the user to the second external electronic device (e.g., a server for a rescue request) connected to the first external electronic device through the second RAT. According to an embodiment, the processor 310 may also transmit the information representing the emergency state of the user and the information on the conscious state of the user to the first external electronic device (e.g., the server for the rescue request).

According to an embodiment, the processor 310 may obtain, based on the first time interval, the first data while the state of the user is the normal state. For example, the first data may represent the temperature associated with a portion (e.g., the head) of the body of the user. Therefore, the processor 310 may obtain, the first data, based on the first time interval, from the second timing to the first timing. In response to identifying (or recognizing) the state of the user as the emergency state, the processor 310 may obtain, the first data, based on a second time interval less than the first time interval, from the first timing in which the state of the user is identified (or recognized) as the emergency state to the third timing after the predefined time from the first timing.

According to an embodiment, the processor 310 may receive, from the second wearable device including an acceleration sensor and a gyroscope sensor, third data related to a motion of the user. The processor 310, based on the first data and the third data, may identify that a fall has occurred to the user. The processor 310, in response to identifying that the fall has occurred to the user, may recognize the state of the user as the emergency state.

In an operation 1140, the processor 310 may identify whether the state of the user is the danger monitoring state, based on that the state of the user is not identified (or recognized) as the emergency state. For example, based on the first data representing a temperature within a second reference range distinct from the first reference range, the processor 310 may identify whether the state of the user is the danger monitoring state.

For example, the processor 310 may identify a second value representing a number of times that the temperature is identified within the second reference range, the temperature being obtained based on the first time interval in the predefined time resource. The processor 310 may identify whether the state of the user is the danger monitoring state, according to whether the second value is greater than or equal to the predefined value.

In an operation 1150, in case that the state of the user is not the danger monitoring state, the processor 310 may perform an operation according to the normal state. For example, the processor 310 may perform the operation according to the normal state, based on identifying that the state of the user is not the danger monitoring state. For example, the processor 310 may identify the state of the user as the normal state, based on identifying that the state of the user is not the emergency state and the danger monitoring state. The processor 310 may perform the operation according to the normal state. As an example, the processor 310 may obtain the data on the motion (or the inertial signal) by using the inertial sensor while the state of the user is the normal state. The inertial sensor may be included in one of the wearable device 300, the second wearable device, and the first external electronic device.

In an operation 1160, in case that the state of the user is the danger monitoring state, the processor 310 may store the second content (e.g., the video, the image, the voice, the call record (or content), the position information of the wearable device 300, and the information on the application being executed) in the memory, provide information that warns of possible danger, and transmit the first request to the second wearable device.

For example, based on identifying that the second value representing a number of times that the temperature is identified within the second reference range, the temperature being obtained based on the first time interval in the predefined time resource is greater than or equal to the predefined value, the processor 310 may identify that the state of the user is the danger monitoring state.

For example, based on recognizing the state of the user as the danger monitoring state, the processor 310 may obtain the second content (e.g., the video, the image, the voice, the call record (or content), the position information of the wearable device 300, and the information on the application being executed) from a fourth timing in which the state of the user is recognized as the danger monitoring state through the camera 330, to a fifth timing after the predefined time from the fourth timing. The processor 310 may store the second content obtained from the fourth timing to the fifth timing. For example, the processor 310 may obtain a fifth content from a sixth timing which is before the predefined time from the fourth timing to the fourth timing. The processor 310 may store the fifth content in the buffer configured in the memory 340. Based on recognizing the state of the user as the danger monitoring state, the processor 310 may remove the fifth content stored in the buffer. The processor 310 may remove the fifth content, and obtain and store the second content, using the camera 330 from the fourth timing to the fifth timing. For example, the processor 310 may store content (e.g., the second content) obtained from the timing (e.g., the fourth timing) when the state of the user is recognized as the danger monitoring state to the predefined time. Therefore, since the buffer stores the content (e.g., the fifth content) obtained from a timing prior to the timing when the state of the user is recognized as the danger monitoring state, the fifth content may be removed from the buffer. The processor 310 may secure capacity of the buffer by removing the fifth content. For example, the processor 310 may provide the information that warns of possible danger, based on recognizing the state of the user as the danger monitoring state. The processor 310 may identify the possible danger. The processor 310 may provide the information that warns of possible danger through sound and/or a screen. For example, the processor 310 may identify that an external object is approaching the user, using the camera 330. The processor 310 may identify information on a size and velocity of the external object approaching the user, using the camera 330. The processor 310 may identify the possible danger to the user based on the information on the size and the velocity of the external object. In response to identifying the possible danger to the user, the processor 310 may recognize the state of the user as the danger monitoring state. The processor 310 may provide information representing that the external object may approach and cause a collision.

According to an embodiment, the processor 310 may obtain, based on the first time interval, the first data while the state of the user is the normal state. For example, the first data may represent the temperature of a portion (e.g., the head) of the body of the user. In response to identifying (or recognizing) the state of the user as the danger monitoring state, the processor 310 may obtain, the first data, based on a third time interval longer than the second time interval, from the fourth timing in which the state of the user is identified (or recognized) as the danger monitoring state to the fifth timing after the predefined time from the fourth timing. For example, the second time interval may be an interval in which the first data is obtained by the processor 310 while the state of the user is the emergency state. Therefore, the processor 310 may obtain the first data based on the first time interval, while the state of the user is the normal state. The processor 310 may obtain the first data, based on the third time interval, while the state of the user is the danger monitoring state. The processor 310 may obtain the first data, based on the second time interval, while the state of the user is the emergency state. The first time interval may be set to be greater than the second time interval. The second time interval may be set to be greater than the third time interval.

According to an embodiment, based on recognizing the state of the user as the danger monitoring state, the processor 310 may maintain an inactive state of the pupil recognition sensor 402 and the brainwave sensor 403. Based on recognizing the state of the user as the danger monitoring state, the processor 310 may refrain from transmitting, to the first external electronic device, the second request.

For example, based on recognizing the state of the user as the danger monitoring state, the processor 310 may transmit, to the second wearable device, the first request to obtain the second data associated with the heart rate of the user. The processor 310 may control to obtain the second data on the heart rate of the user by transmitting the first request to the second wearable device.

In an operation 1170, the processor 310 may receive, from the second wearable device, the second data in response to the first request. For example, the processor 310 may receive, from the second wearable device, the second data on the heart rate of the user in response to the first request.

In an operation 1180, the processor 310 may identify whether the state of the user is changed from the danger monitoring state to the emergency state. For example, the processor 310 may identify, based on the second data, whether the state of the user is changed from the danger monitoring state to the emergency state.

According to an embodiment, the processor 310 may set the first data and the second data as an input value of a predefined model indicated by a plurality of parameters stored in the memory 340. For example, the first data may represent the temperature associated with a portion (e.g., the head) of the body of the user. The second data may represent the heart rate of the user. For example, the predefined model may be indicated by a plurality of parameters related to a neural network. The predefined model may include a set of parameters related to the neural network. For example, the parameters related to the neural network may represent a plurality of nodes included in the neural network and/or weight assigned to a connection between the plurality of nodes.

The processor 310 may identify whether the state of the user is changed from the danger monitoring state to the emergency state by identifying an output value of the predefined model. According to an embodiment, the processor 310 may train the predefined model based on the first data and the second data.

For example, in case that the state of the user is changed from the danger monitoring state to the emergency state, the processor 310 may operate according to the operation 1130. For example, in case that the state of the user is not changed from the danger monitoring state to the emergency state, the processor 310 may operate according to the operation 1150. As an example, based on identifying that the state of the user is maintained in the danger monitoring state for a designated time, the processor 310 may change the state of the user from the danger monitoring state to the normal state. After changing the state of the user from the danger monitoring state to the normal state, the processor 310 may operate according to the operation 1150.

According to an embodiment, the processor 310 may identify a trend (or a graph) related to a body temperature of the user. The processor 310 may determine, based on the trend related to the body temperature of the user, the first reference range and the second reference range. The trend related to the body temperature of the user may be changed according to at least one of an age, a BMI, and a gender. Accordingly, the processor 310 may identify the trend related to the body temperature of the user, and determine, based on the identified trend, the first reference range for identifying the emergency state and the second reference range for identifying the danger monitoring state.

According to an embodiment, the processor 310 may recognize, according to a user input, the state of the user.

For example, the processor 310 may recognize, based on a first user input, the state of the user as the danger monitoring state. The processor 310, based on recognizing the state of the user as the danger monitoring state, may set a mode related to the wearable device 300, the second wearable device, and the first external electronic device, to a first mode. The first mode may include a mode in which a first set of sensors used for identifying the possible danger are set to activate among a plurality of sensors (or components) included in the wearable device 300, the second wearable device, and the first external electronic device.

For example, the processor 310, based on a second user input, may recognize the state of the user as the emergency state. The processor 310 may set, based on recognizing the state of the user as the emergency state, the mode related to the wearable device 300, the second wearable device, and the first external electronic device to a second mode. The second mode may include a mode in which the first set of sensors and a second set of sensors used for obtaining information on the conscious state of the user are set to activate among the plurality of sensors(or components) included in the wearable device 300, the second wearable device, and the first external electronic device.

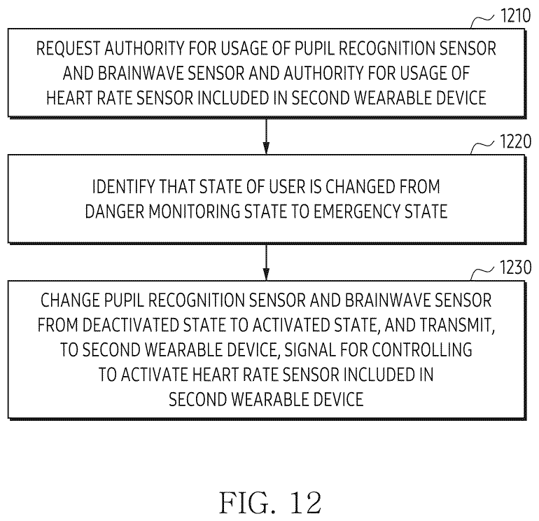

FIG. 12 is a flowchart illustrating an operation of a wearable device according to an embodiment of the disclosure.

Referring to FIG. 12, in an operation 1210, a processor 310 may request authority for usage of a pupil recognition sensor 402 and a brainwave sensor 403 and authority for usage of a heart rate sensor included in a second wearable device. For example, the processor 310, based on recognizing a state of a user as a danger monitoring state, may request the authority for the usage of the pupil recognition sensor 402 and the brainwave sensor 403 and the authority for the usage of the heart rate sensor included in the second wearable device.

For example, the processor 310, using at least one of a wearable device 300 and the second wearable device, may request the authority for the usage of the pupil recognition sensor 402 and the brainwave sensor 403 included in the wearable device 300 and the heart rate sensor included in the second wearable device. As an example, the processor 310, using the wearable device 300, may request the authority for the usage of the heart rate sensor included in the second wearable device. As an example, the processor 310, using the second wearable device, may request the authority for the usage of the pupil recognition sensor 402 and the brainwave sensor 403 included in the wearable device 300.

In an operation 1220, the processor 310 may identify that the state of the user is changed from the danger monitoring state to an emergency state. For example, after obtaining the authority for the usage of the pupil recognition sensor 402 and the brainwave sensor 403 included in the wearable device 300 and the authority for the usage of the heart rate sensor included in the second wearable device, the processor 310 may identify that the state of the user is changed from the danger monitoring state to the emergency state.

In an operation 1230, the processor 310 may change the pupil recognition sensor 402 and the brainwave sensor 403 from a deactivated state to an activated state, and transmit, to the second wearable device, a signal for controlling to activate the heart rate sensor included in the second wearable device.

For example, the processor 310, based on the authority for the usage of the pupil recognition sensor 402 and the brainwave sensor 403, may change the pupil recognition sensor 402 and the brainwave sensor 403 from the deactivated state to the activated state.

For example, the processor 310, based on the authority for the usage of the heart rate sensor included in the second wearable device, may transmit, to the second wearable device, the signal for controlling to activate the heart rate sensor included in the second wearable device. The second wearable device, based on the signal, may change the heart rate sensor from the deactivated state to the activated state. The second wearable device may obtain second data on the heart rate of the user, using the heart rate sensor. The second wearable device may transmit the second data on the heart rate of the user to the wearable device 300.

According to an embodiment, a wearable device (e.g., a wearable device 300) may include a temperature sensor (e.g., a temperature sensor 401), a pupil recognition sensor (e.g., a pupil recognition sensor 402), a brainwave sensor (e.g., a brainwave sensor 403), a camera (e.g., a camera 330), memory (e.g., memory 340), communication circuitry (e.g., communication circuitry 350), and a processor (e.g., a processor 310) operatively coupled with the temperature sensor, the pupil recognition sensor, the brainwave sensor, the camera, and the memory. The processor may be configured to obtain, through the temperature sensor, first data representing a temperature associated with a head of a user, contacted with the wearable device. The processor may be configured to recognize, based on the first data representing the temperature within a first reference range, a state of the user as an emergency state. The processor may be configured to, based on recognizing the state of the user as the emergency state, store, in the memory, first content obtained from a second timing which is before predefined time from a first timing in which the state of the user is recognized as the emergency state through the camera, to a third timing after the predefined time from the first timing and information on a conscious state of the user obtained from the first timing to the third timing using the pupil recognition sensor and the brainwave sensor activated in response to the recognition of the emergency state, transmit, to a second wearable device contacted with a wrist, a first request to obtain second data associated with a heart rate of the user, and transmit, to a first external electronic device, a second request to transmit information representing the emergency state of the user and the information on the conscious state of the user to a second external electronic device. The processor may be configured to, based on the first data representing the temperature within a second reference range distinct from the first reference range, recognize the state of the user as a danger monitoring state. The processor may be configured to, based on recognizing the state of the user as the danger monitoring state, store, in the memory, second content obtained from a fourth timing in which the state of the user is recognized as the danger monitoring state through the camera, to a fifth timing after the predefined time from the fourth timing, provide information that warns of possible danger, transmit, to the second wearable device, the first request, receive, from the second wearable device, the second data in response to the first request, and identify, using the first data and the second data, whether the state of the user is changed from the danger monitoring state to the emergency state.

According to an embodiment, the processor may be configured to obtain, based on a first time interval, the first data from the second timing to the first timing. The processor may be configured to, in response to recognizing the state of the user as the emergency state, obtain, based on a second time interval less than the first time interval, the first data from the first timing to the third timing.

According to an embodiment, the processor may be configured to, in response to recognizing the state of the user as the danger monitoring state, obtain, based on a third time interval less than the first time interval and longer than the second time interval, the first data from the fourth timing to the fifth timing.

According to an embodiment, the processor may be configured to, based on the first data, identify a trend related to a body temperature of the user. The processor may be configured to determine, based on the trend related to the body temperature of the user, the first reference range and the second reference range.

According to an embodiment, the processor may be configured to, based on recognizing the state of the user as the danger monitoring state, maintain an inactive state of the pupil recognition sensor and the brainwave sensor, and refrain from transmitting, to the first external electronic device, the second request.

According to an embodiment, the processor may be configured to store in a buffer configured in the memory, third content obtained using the camera from the second timing to the first timing. The processor may be configured to, in response to recognizing the state of the user as the emergency state, obtain the first content by combining the third content stored in the buffer and fourth content obtained from the first timing to the third timing.

According to an embodiment, the processor may be configured to store in a buffer configured in the memory, fifth content obtained from a sixth timing which is before the predefined time from the fourth timing to the fourth timing. The processor may be configured to, based on recognizing the state of the user as the danger monitoring state, remove the fifth content stored in the buffer. The processor may be configured to obtain, using the camera, the second content from the fourth timing to the fifth timing.

According to an embodiment, the processor may be configured to, based on recognizing the state of the user as the danger monitoring state, request authority for usage of the pupil recognition sensor and the brainwave sensor and authority for usage of a heart rate sensor included in the second wearable device. The processor may be configured to, after obtaining the authority for usage of the pupil recognition sensor and the brainwave sensor and the authority for usage of the heart rate sensor included in the second wearable device, identify that the state of the user is changed from the danger monitoring state to the emergency state. The processor may be configured to, in response to identifying that the state of the user is changed from the danger monitoring state to the emergency state, change the pupil recognition sensor and the brainwave sensor from a deactivated state to an activated state, and transmit, to the second wearable device, a signal for controlling to activate the heart rate sensor included in the second wearable device.

According to an embodiment, the processor may be configured to recognize, based on a first user input, the state of the user as the danger monitoring state. The processor may be configured to, based on recognizing the state of the user as the danger monitoring state, set a mode related to the wearable device, the second wearable device, and the first external electronic device, to a first mode. The processor may be configured to, while the wearable device is operating in the first mode, based on a second user input distinct from the first user input, recognize the state of the user as the emergency state. The processor may be configured to set, based on recognizing the state of the user as the emergency state, the mode to a second mode distinct from the first mode. The first mode may include a mode in which a first set of sensors used for identifying the possible danger are set to activate among a plurality of sensors included in the wearable device, the second wearable device, and the first external electronic device. The second mode may include a mode in which the first set of sensors and a second set of sensors used for obtaining information on a conscious state of the user are set to activate among the plurality of sensors.

According to an embodiment, the processor may be configured to set the first data and the second data as an input value of a predefined model indicated by a plurality of parameters stored in the memory. The processor may be configured to identify whether the state of the user is changed from the danger monitoring state to the emergency state by identifying an output value of the predefined model.

According to an embodiment, the processor may be configured to train the predefined model based on the first data and the second data.

According to an embodiment, the processor may be configured to identify a first value representing a number of times that the temperature is identified within the first reference range, the temperature being obtained based on a first time interval in predefined time resource. The processor may be configured to recognize the state of the user as the emergency state in response to identifying that the first value is greater than or equal to a predefined value. The processor may be configured to identify a second value representing a number of times that the temperature is identified within the second reference range, the temperature being obtained based on the first time interval in the predefined time resource. The processor may be configured to recognize the state of the user as the danger monitoring state in response to identifying that the second value is greater than or equal to the predefined value.

According to an embodiment, the camera may be disposed to correspond to a gaze of the user. The processor may be configured to identify, using the camera, that the external object is approaching to the user. The processor may be configured to identify, using the camera, information on a size and velocity of the external object approaching to the user. The processor may be configured to, based on the size and the velocity of the external object, identify the possible danger to the user. The processor may be configured to, in response to identifying the possible danger to the user, recognize the state of the user as the danger monitoring state.

According to an embodiment, the processor may be configured to receive, from the second wearable device including an acceleration sensor and a gyroscope sensor, third data related to a motion of the user. The processor may be configured to, based on the first data and the third data, identify that a fall has occurred to the user. The processor may be configured to, in response to identifying that the fall has occurred to the user, recognize the state of the user as the emergency state.

According to an embodiment, the first content may include at least one of a video, an image, a voice, a call record, position information of a wearable device, and information on an application being executed. The second content may include at least one of the video, the image, the voice, the call record, the position information of the wearable device, and the information on the application being executed.

According to an embodiment, a method of a wearable device may include obtaining, through a temperature sensor of the wearable device, first data representing a temperature associated with a head of a user, contacted with the wearable device. The operation may include recognizing, based on the first data representing the temperature within a first reference range, a state of the user as an emergency state. The operation may include, based on recognizing the state of the user as the emergency state, storing, in memory of the wearable device, first content obtained from a second timing which is before predefined time from a first timing in which the state of the user is recognized as the emergency state through a camera of the wearable device, to a third timing after the predefined time from the first timing and information on a conscious state of the user obtained from the first timing to the third timing using a pupil recognition sensor of the wearable device and a brainwave sensor of the wearable device activated in response to the recognition of the emergency state, transmitting, to a second wearable device contacted with a wrist, a first request to obtain second data associated with a heart rate of the user, and transmitting, to a first external electronic device, a second request to transmit information representing the emergency state of the user and the information on the conscious state of the user to a second external electronic device. The operation may include, based on the first data representing the temperature within a second reference range distinct from the first reference range, recognizing the state of the user as a danger monitoring state. The operation may include, based on recognizing the state of the user as the danger monitoring state, storing, in the memory, second content obtained from a fourth timing in which the state of the user is recognized as the danger monitoring state through the camera, to a fifth timing after the predefined time from the fourth timing, providing information that warns of possible danger, transmitting, to the second wearable device, the first request, receiving, from the second wearable device, the second data in response to the first request, and identifying, using the first data and the second data, whether the state of the user is changed from the danger monitoring state to the emergency state.

According to an embodiment, the method may include obtaining, based on a first time interval, the first data from the second timing to the first timing. The operation may include, in response to recognizing the state of the user as the emergency state, obtaining, based on a second time interval less than the first time interval, the first data from the first timing to the third timing.

According to an embodiment, the method may include, in response to recognizing the state of the user as the danger monitoring state, obtaining, based on a third time interval less than the first time interval and longer than the second time interval, the first data from the fourth timing to the fifth timing.

According to an embodiment, the method may include, based on recognizing the state of the user as the danger monitoring state, maintaining an inactive state of the pupil recognition sensor and the brainwave sensor, and refraining from transmitting, to the first external electronic device, the second request.

According to an embodiment, the method may include storing in a buffer configured in the memory, third content obtained using the camera from the second timing to the first timing. The operation may include, in response to recognizing the state of the user as the emergency state, obtaining the first content by combining the third content stored in the buffer and fourth content obtained from the first timing to the third timing.

According to an embodiment, the method may include storing in a buffer configured in the memory, fifth content obtained from a sixth timing which is before the predefined time from the fourth timing to the fourth timing. The operation may include, based on recognizing the state of the user as the danger monitoring state, removing the fifth content stored in the buffer. The operation may include obtaining, using the camera, the second content from the fourth timing to the fifth timing.

According to an embodiment, a wearable device (e.g., a wearable device 300) may include at least one sensor (e.g., a sensor 320), a camera (e.g., a camera 330), memory (e.g., memory 340), and a processor (e.g., a processor 310), wherein the processor may be configured to obtain, through the at least one sensor, first data associated with a body of a user, obtained from a first portion of the body of the user contacted with the wearable device, recognize a state of the user as a first state that is an emergency state, based on that the first data satisfies a first condition, store, based on recognizing the first state as the first state, in the memory, first content obtained from a second timing which is before predefined time from a first timing in which the state of the user is recognized as the emergency state through the camera, to a third timing after the predefined time from the first timing, recognize the state as a second state identifying a change to the emergency state based on the first data satisfying a second condition distinct from the first condition, and store, in the memory, based on recognizing the state as the second state, second content obtained from a fourth timing in which the state of the user is recognized as the danger monitoring state through the camera, to a fifth timing after the predefined time from the fourth timing.

According to an embodiment, a wearable device comprises a temperature sensor, a pupil recognition sensor, a brainwave sensor, a camera, memory storing instructions, comprising one or more storage media, communication circuitry, and one or more processors comprising processing circuitry. The instructions that, when executed by the one or more processors individually or collectively, cause the wearable device to obtain, through the temperature sensor, first data representing a temperature associated with a head of a user, contacted with the wearable device, recognize, based on the first data representing the temperature within a first reference range, a state of the user as an emergency state, based on recognizing the state of the user as the emergency state, store, in the memory, first content obtained from a second timing which is before predefined time from a first timing in which the state of the user is recognized as the emergency state through the camera, to a third timing after the predefined time from the first timing and information on a conscious state of the user obtained from the first timing to the third timing using the pupil recognition sensor or the brainwave sensor activated in response to the recognition of the emergency state, transmit, to a second wearable device contacted with a wrist, a first request to obtain second data associated with a heart rate of the user, transmit, to a first external electronic device, a second request to transmit information representing the emergency state of the user and the information on the conscious state of the user to a second external electronic device, based on the first data representing the temperature within a second reference range distinct from the first reference range, recognize the state of the user as a danger monitoring state, and based on recognizing the state of the user as the danger monitoring state store, in the memory, second content obtained from a fourth timing in which the state of the user is recognized as the danger monitoring state through the camera, to a fifth timing after the predefined time from the fourth timing, provide information that warns of possible danger, transmit, to the second wearable device, the first request, receive, from the second wearable device, the second data in response to the first request, and identify, using the first data and the second data, whether the state of the user is changed from the danger monitoring state to the emergency state.

According to an embodiment, the instructions that, when executed by the one or more processors individually or collectively, cause the wearable device to obtain, based on a first time interval, the first data from the second timing to the first timing, and in response to recognizing the state of the user as the emergency state, obtain, based on a second time interval less than the first time interval, the first data from the first timing to the third timing.

According to an embodiment, the instructions that, when executed by the one or more processors individually or collectively, cause the wearable device to in response to recognizing the state of the user as the danger monitoring state, obtain, based on a third time interval less than the first time interval and longer than the second time interval, the first data from the fourth timing to the fifth timing.

According to an embodiment, the instructions that, when executed by the one or more processors individually or collectively, cause the wearable device to identify a trend related to a body temperature of the user, and determine, based on the trend related to the body temperature of the user, the first reference range and the second reference range.

According to an embodiment, the instructions that, when executed by the one or more processors individually or collectively, cause the wearable device to, based on recognizing the state of the user as the danger monitoring state, maintain an inactive state of the pupil recognition sensor and the brainwave sensor, and refrain from transmitting, to the first external electronic device, the second request.

According to an embodiment, the instructions that, when executed by the one or more processors individually or collectively, cause the wearable device to store in a buffer configured on the memory, third content obtained using the camera from the second timing to the first timing, and in response to recognizing the state of the user as the emergency state, obtain the first content by combining the third content stored in the buffer and fourth content obtained from the first timing to the third timing.

According to an embodiment, the instructions that, when executed by the one or more processors individually or collectively, cause the wearable device to store in a buffer configured in the memory, fifth content obtained from a sixth timing which is before the predefined time from the fourth timing to the fourth timing, based on recognizing the state of the user as the danger monitoring state, remove the fifth content stored in the buffer, and obtain, using the camera, the second content from the fourth timing to the fifth timing.

According to an embodiment, the instructions that, when executed by the one or more processors individually or collectively, cause the wearable device to, based on recognizing the state of the user as the danger monitoring state, request authority for usage of the pupil recognition sensor and the brainwave sensor and authority for usage of a heart rate sensor comprised in the second wearable device, after obtaining the authority for usage of the pupil recognition sensor and the brainwave sensor and the authority for usage of the heart rate sensor comprised in the second wearable device, identify that the state of the user is changed from the danger monitoring state to the emergency state, and in response to identifying that the state of the user is changed from the danger monitoring state to the emergency state, change the pupil recognition sensor and the brainwave sensor from a deactivated state to an activated state, and transmit, to the second wearable device, a signal for controlling to activate the heart rate sensor comprised in the second wearable device.

According to an embodiment, the instructions that, when executed by the one or more processors individually or collectively, cause the wearable device to recognize, based on a first user input, the state of the user as the danger monitoring state, based on recognizing the state of the user as the danger monitoring state, set a mode related to the wearable device, the second wearable device, and the first external electronic device, to a first mode, while the wearable device is operating in the first mode, based on a second user input distinct from the first user input, recognize the state of the user as the emergency state, and set, based on recognizing the state of the user as the emergency state, the mode to a second mode distinct from the first mode. The first mode comprises a mode in which a first set of sensors used for identifying the possible danger are set to activate among a plurality of sensors included in the wearable device, the second wearable device, and the first external electronic device. The second mode comprises a mode in which the first set of sensors and a second set of sensors used for obtaining information on a conscious state of the user are set to activate among the plurality of sensors.

According to an embodiment, the instructions that, when executed by the one or more processors individually or collectively, cause the wearable device to set the first data and the second data as an input value of a predefined model indicated by a plurality of parameters stored in the memory, and identify whether the state of the user is changed from the danger monitoring state to the emergency state by identifying an output value of the predefined model.

According to an embodiment, the instructions that, when executed by the one or more processors individually or collectively, cause the wearable device to train the predefined model based on the first data and the second data.

According to an embodiment, the instructions that, when executed by the one or more processors individually or collectively, cause the wearable device to identify a first value representing a number of times that the temperature is identified within the first reference range, the temperature being obtained based on a first time interval in predefined time resource, recognize the state of the user as the emergency state in response to identifying that the first value is greater than or equal to a predefined value, identify a second value representing a number of times that the temperature is identified within the second reference range, the temperature being obtained based on the first time interval in the predefined time resource, and recognize the state of the user as the danger monitoring state in response to identifying that the second value is greater than or equal to the predefined value.

According to an embodiment, the camera is disposed to correspond to a gaze of the user. The instructions that, when executed by the one or more processors individually or collectively, cause the wearable device to identify, using the camera, that an external object is approaching to the user, identify, using the camera, information on a size and velocity of the external object approaching to the user, based on the size and the velocity of the external object, identify the possible danger to the user, and in response to identifying the possible danger to the user, recognize the state of the user as the danger monitoring state.

According to an embodiment, the instructions that, when executed by the one or more processors individually or collectively, cause the wearable device to receive, from the second wearable device comprising an acceleration sensor and a gyroscope sensor, third data related to a motion of the user, based on the first data and the third data, identify that a fall has occurred to the user, and in response to identifying that the fall has occurred to the user, identify the state of the user as the emergency state.

According to an embodiment, a method performed by a wearable device, comprises obtaining, by the wearable device through a temperature sensor of the wearable device, first data representing a temperature associated with a head of a user, contacted with the wearable device, recognizing, by the wearable device, based on the first data representing the temperature within a first reference range, a state of the user as an emergency state, and based on recognizing the state of the user as the emergency state, storing, by the wearable device in memory of the wearable device, first content obtained from a second timing which is before predefined time from a first timing in which the state of the user is recognized as the emergency state through a camera of the wearable device, to a third timing after the predefined time from the first timing and information on a conscious state of the user obtained from the first timing to the third timing using a pupil recognition sensor of the wearable device and a brainwave sensor of the wearable device activated in response to the recognition of the emergency state, transmitting, by the wearable device to a second wearable device contacted with a wrist, a first request to obtain second data associated with a heart rate of the user, transmitting, by the wearable device to a first external electronic device, a second request to transmit information representing the emergency state of the user and the information on the conscious state of the user to a second external electronic device, based on the first data representing the temperature within a second reference range distinct from the first reference range, recognizing, by the wearable device, the state of the user as a danger monitoring state, and based on recognizing the state of the user as the danger monitoring state, storing, by the wearable device in the memory, second content obtained from a fourth timing in which the state of the user is recognized as the danger monitoring state through the camera, to a fifth timing after the predefined time from the fourth timing, providing, by the wearable device, information that warns of possible danger, transmitting, by the wearable device to the second wearable device, the first request, receiving, by the wearable device from the second wearable device, the second data in response to the first request, and identifying, by the wearable device, using the first data and the second data, whether the state of the user is changed from the danger monitoring state to the emergency state.

According to an embodiment, the method comprises obtaining, based on a first time interval, the first data from the second timing to the first timing, and in response to recognizing the state of the user as the emergency state, obtaining, based on a second time interval less than the first time interval, the first data from the first timing to the third timing.

According to an embodiment, the method comprises, in response to recognizing the state of the user as the danger monitoring state, obtaining, based on a third time interval less than the first time interval and longer than the second time interval, the first data from the fourth timing to the fifth timing.

According to an embodiment, the method comprises identifying a trend related to a body temperature of the user, and determining, based on the trend related to the body temperature of the user, the first reference range and the second reference range.

According to an embodiment, one or more non-transitory computer-readable storage media store one or more computer programs including computer-executable instructions. The computer-executable instructions that, when executed by one or more processors of an wearable device individually or collectively, cause the wearable device to perform operations. The operations comprises obtaining, by the wearable device through a temperature sensor of the wearable device, first data representing a temperature associated with a head of a user, contacted with the wearable device, recognizing, by the wearable device, based on the first data representing the temperature within a first reference range, a state of the user as an emergency state, and based on recognizing the state of the user as the emergency state, storing, by the wearable device in memory of the wearable device, first content obtained from a second timing which is before predefined time from a first timing in which the state of the user is recognized as the emergency state through a camera of the wearable device, to a third timing after the predefined time from the first timing and information on a conscious state of the user obtained from the first timing to the third timing using a pupil recognition sensor of the wearable device and a brainwave sensor of the wearable device activated in response to the recognition of the emergency state, transmitting, by the wearable device to a second wearable device contacted with a wrist, a first request to obtain second data associated with a heart rate of the user, transmitting, by the wearable device to a first external electronic device, a second request to transmit information representing the emergency state of the user and the information on the conscious state of the user to a second external electronic device, based on the first data representing the temperature within a second reference range distinct from the first reference range, recognizing, by the wearable device, the state of the user as a danger monitoring state, and based on recognizing the state of the user as the danger monitoring state, storing, by the wearable device in the memory, second content obtained from a fourth timing in which the state of the user is recognized as the danger monitoring state through the camera, to a fifth timing after the predefined time from the fourth timing, providing, by the wearable device, information that warns of possible danger, transmitting, by the wearable device to the second wearable device, the first request, receiving, by the wearable device from the second wearable device, the second data in response to the first request, and identifying, by the wearable device, using the first data and the second data, whether the state of the user is changed from the danger monitoring state to the emergency state.

According to an embodiment, the operations comprise obtaining, based on a first time interval, the first data from the second timing to the first timing, and in response to recognizing the state of the user as the emergency state, obtaining, based on a second time interval less than the first time interval, the first data from the first timing to the third timing.

According to the above-described embodiments, there is an effect capable of increasing accuracy of data on a user obtained using a wearable device. According to the above-described embodiments, there is an effect of increasing peripheral cognition sense of the user caused by wearing the wearable device. According to the above-described embodiments, there is an effect of identifying a dangerous situation in advance, and providing a notification of possible danger, with a wearable device. According to the above-described embodiments, there is an effect of identifying a state of the user, based on the data on the user obtained from a second external electronic device connected to the wearable device as well as the wearable device.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” or “connected with” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between a case in which data is semi-permanently stored in the storage medium and a case in which the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been shown and described with reference to various embodiments thereof, it will be understood by those skilled in the art that various changes in form and details may be made therein without departing from the spirit and scope of the disclosure as defined by the appended claims and their equivalents.

No claim element is to be construed under the provisions of 35 U.S.C. § 112, sixth paragraph, unless the element is expressly recited using the phrase “means for” or “means”.

What is claimed is:

1. A wearable device comprising:
a temperature sensor;
a pupil recognition sensor;
a brainwave sensor;
a camera;
memory storing instructions, comprising one or more storage media;
communication circuitry; and
one or more processors comprising processing circuitry,
wherein the instructions that, when executed by the one or more processors individually or collectively, cause the wearable device to:
obtain, through the temperature sensor, first data representing a temperature associated with a head of a user, contacted with the wearable device,
recognize, based on the first data representing the temperature within a first reference range, a state of the user as an emergency state,
based on recognizing the state of the user as the emergency state:
store, in the memory, first content obtained from a second timing which is before predefined time from a first timing in which the state of the user is recognized as the emergency state through the camera, to a third timing after the predefined time from the first timing and information on a conscious state of the user obtained from the first timing to the third timing using the pupil recognition sensor or the brainwave sensor activated in response to the recognition of the emergency state,
transmit, to a second wearable device contacted with a wrist, a first request to obtain second data associated with a heart rate of the user,
transmit, to a first external electronic device, a second request to transmit information representing the emergency state of the user and the information on the conscious state of the user to a second external electronic device,
based on the first data representing the temperature within a second reference range distinct from the first reference range, recognize the state of the user as a danger monitoring state, and
based on recognizing the state of the user as the danger monitoring state:
store, in the memory, second content obtained from a fourth timing in which the state of the user is recognized as the danger monitoring state through the camera, to a fifth timing after the predefined time from the fourth timing,
provide information that warns of possible danger,
transmit, to the second wearable device, the first request,
receive, from the second wearable device, the second data in response to the first request, and
identify, using the first data and the second data, whether the state of the user is changed from the danger monitoring state to the emergency state.

2. The wearable device of claim 1,
wherein the instructions that, when executed by the one or more processors individually or collectively, cause the wearable device to:
obtain, based on a first time interval, the first data from the second timing to the first timing, and
in response to recognizing the state of the user as the emergency state, obtain, based on a second time interval less than the first time interval, the first data from the first timing to the third timing.

3. The wearable device of claim 2, wherein the instructions that, when executed by the one or more processors individually or collectively, cause the wearable device to:
in response to recognizing the state of the user as the danger monitoring state, obtain, based on a third time interval less than the first time interval and longer than the second time interval, the first data from the fourth timing to the fifth timing.

4. The wearable device of claim 1, wherein the instructions that, when executed by the one or more processors individually or collectively, cause the wearable device to:
identify a trend related to a body temperature of the user, and
determine, based on the trend related to the body temperature of the user, the first reference range and the second reference range.

5. The wearable device of claim 1, wherein the instructions that, when executed by the one or more processors individually or collectively, cause the wearable device to:
based on recognizing the state of the user as the danger monitoring state,
maintain an inactive state of the pupil recognition sensor and the brainwave sensor, and
refrain from transmitting, to the first external electronic device, the second request.

6. The wearable device of claim 1, wherein the instructions that, when executed by the one or more processors individually or collectively, cause the wearable device to:
store in a buffer configured on the memory, third content obtained using the camera from the second timing to the first timing, and
in response to recognizing the state of the user as the emergency state, obtain the first content by combining the third content stored in the buffer and fourth content obtained from the first timing to the third timing.

7. The wearable device of claim 1, wherein the instructions that, when executed by the one or more processors individually or collectively, cause the wearable device to:
store in a buffer configured in the memory, fifth content obtained from a sixth timing which is before the predefined time from the fourth timing to the fourth timing,
based on recognizing the state of the user as the danger monitoring state, remove the fifth content stored in the buffer, and
obtain, using the camera, the second content from the fourth timing to the fifth timing.

8. The wearable device of claim 1, wherein the instructions that, when executed by the one or more processors individually or collectively, cause the wearable device to:
based on recognizing the state of the user as the danger monitoring state, request authority for usage of the pupil recognition sensor and the brainwave sensor and authority for usage of a heart rate sensor comprised in the second wearable device,
after obtaining the authority for usage of the pupil recognition sensor and the brainwave sensor and the authority for usage of the heart rate sensor comprised in the second wearable device, identify that the state of the user is changed from the danger monitoring state to the emergency state, and
in response to identifying that the state of the user is changed from the danger monitoring state to the emergency state:
change the pupil recognition sensor and the brainwave sensor from a deactivated state to an activated state, and
transmit, to the second wearable device, a signal for controlling to activate the heart rate sensor comprised in the second wearable device.

9. The wearable device of claim 1, wherein the instructions that, when executed by the one or more processors individually or collectively, cause the wearable device to:
recognize, based on a first user input, the state of the user as the danger monitoring state,
based on recognizing the state of the user as the danger monitoring state, set a mode related to the wearable device, the second wearable device, and the first external electronic device, to a first mode,
while the wearable device is operating in the first mode, based on a second user input distinct from the first user input, recognize the state of the user as the emergency state, and
set, based on recognizing the state of the user as the emergency state, the mode to a second mode distinct from the first mode,
wherein the first mode comprises a mode in which a first set of sensors used for identifying the possible danger are set to activate among a plurality of sensors included in the wearable device, the second wearable device, and the first external electronic device, and
wherein the second mode comprises a mode in which the first set of sensors and a second set of sensors used for obtaining information on a conscious state of the user are set to activate among the plurality of sensors.

10. The wearable device of claim 1, wherein the instructions that, when executed by the one or more processors individually or collectively, cause the wearable device to:
set the first data and the second data as an input value of a predefined model indicated by a plurality of parameters stored in the memory, and
identify whether the state of the user is changed from the danger monitoring state to the emergency state by identifying an output value of the predefined model.

11. The wearable device of claim 10, wherein the instructions that, when executed by the one or more processors individually or collectively, cause the wearable device to:
train the predefined model based on the first data and the second data.

12. The wearable device of claim 10, wherein the instructions that, when executed by the one or more processors individually or collectively, cause the wearable device to:
identify a first value representing a number of times that the temperature is identified within the first reference range, the temperature being obtained based on a first time interval in predefined time resource,
recognize the state of the user as the emergency state in response to identifying that the first value is greater than or equal to a predefined value,
identify a second value representing a number of times that the temperature is identified within the second reference range, the temperature being obtained based on the first time interval in the predefined time resource, and
recognize the state of the user as the danger monitoring state in response to identifying that the second value is greater than or equal to the predefined value.

13. The wearable device of claim 1, wherein the camera is disposed to correspond to a gaze of the user, and
wherein the instructions that, when executed by the one or more processors individually or collectively, cause the wearable device to:
identify, using the camera, that an external object is approaching to the user,
identify, using the camera, information on a size and velocity of the external object approaching to the user,
based on the size and the velocity of the external object, identify the possible danger to the user, and
in response to identifying the possible danger to the user, recognize the state of the user as the danger monitoring state.

14. The wearable device of claim 1, wherein the instructions that, when executed by the one or more processors individually or collectively, cause the wearable device to:
receive, from the second wearable device comprising an acceleration sensor and a gyroscope sensor, third data related to a motion of the user,
based on the first data and the third data, identify that a fall has occurred to the user, and
in response to identifying that the fall has occurred to the user, identify the state of the user as the emergency state.

15. A method performed by a wearable device, comprising:
obtaining, by the wearable device through a temperature sensor of the wearable device, first data representing a temperature associated with a head of a user, contacted with the wearable device;
recognizing, by the wearable device, based on the first data representing the temperature within a first reference range, a state of the user as an emergency state; and
based on recognizing the state of the user as the emergency state:
storing, by the wearable device in memory of the wearable device, first content obtained from a second timing which is before predefined time from a first timing in which the state of the user is recognized as the emergency state through a camera of the wearable device, to a third timing after the predefined time from the first timing and information on a conscious state of the user obtained from the first timing to the third timing using a pupil recognition sensor of the wearable device and a brainwave sensor of the wearable device activated in response to the recognition of the emergency state,
transmitting, by the wearable device to a second wearable device contacted with a wrist, a first request to obtain second data associated with a heart rate of the user,
transmitting, by the wearable device to a first external electronic device, a second request to transmit information representing the emergency state of the user and the information on the conscious state of the user to a second external electronic device,
based on the first data representing the temperature within a second reference range distinct from the first reference range, recognizing, by the wearable device, the state of the user as a danger monitoring state, and
based on recognizing the state of the user as the danger monitoring state:
storing, by the wearable device in the memory, second content obtained from a fourth timing in which the state of the user is recognized as the danger monitoring state through the camera, to a fifth timing after the predefined time from the fourth timing,
providing, by the wearable device, information that warns of possible danger,
transmitting, by the wearable device to the second wearable device, the first request,
receiving, by the wearable device from the second wearable device, the second data in response to the first request, and
identifying, by the wearable device, using the first data and the second data, whether the state of the user is changed from the danger monitoring state to the emergency state.

16. The method of claim 15, further comprising:
obtaining, based on a first time interval, the first data from the second timing to the first timing; and
in response to recognizing the state of the user as the emergency state, obtaining, based on a second time interval less than the first time interval, the first data from the first timing to the third timing.

17. The method of claim 16, further comprising:
in response to recognizing the state of the user as the danger monitoring state, obtaining, based on a third time interval less than the first time interval and longer than the second time interval, the first data from the fourth timing to the fifth timing.

18. The method of claim 15, further comprising:
identifying a trend related to a body temperature of the user; and
determining, based on the trend related to the body temperature of the user, the first reference range and the second reference range.

19. One or more non-transitory computer-readable storage media storing one or more computer programs including computer-executable instructions that, when executed by one or more processors of a wearable device individually or collectively, cause the wearable device to perform operations, the operations comprising:
obtaining, by the wearable device through a temperature sensor of the wearable device, first data representing a temperature associated with a head of a user, contacted with the wearable device;
recognizing, by the wearable device, based on the first data representing the temperature within a first reference range, a state of the user as an emergency state; and
based on recognizing the state of the user as the emergency state:
storing, by the wearable device in memory of the wearable device, first content obtained from a second timing which is before predefined time from a first timing in which the state of the user is recognized as the emergency state through a camera of the wearable device, to a third timing after the predefined time from the first timing and information on a conscious state of the user obtained from the first timing to the third timing using a pupil recognition sensor of the wearable device and a brainwave sensor of the wearable device activated in response to the recognition of the emergency state,
transmitting, by the wearable device to a second wearable device contacted with a wrist, a first request to obtain second data associated with a heart rate of the user,
transmitting, by the wearable device to a first external electronic device, a second request to transmit information representing the emergency state of the user and the information on the conscious state of the user to a second external electronic device,
based on the first data representing the temperature within a second reference range distinct from the first reference range, recognizing, by the wearable device, the state of the user as a danger monitoring state, and
based on recognizing the state of the user as the danger monitoring state:
storing, by the wearable device in the memory, second content obtained from a fourth timing in which the state of the user is recognized as the danger monitoring state through the camera, to a fifth timing after the predefined time from the fourth timing,
providing, by the wearable device, information that warns of possible danger,
transmitting, by the wearable device to the second wearable device, the first request,
receiving, by the wearable device from the second wearable device, the second data in response to the first request, and
identifying, by the wearable device, using the first data and the second data, whether the state of the user is changed from the danger monitoring state to the emergency state.

20. The one or more non-transitory computer-readable storage media of claim 19, the operations further comprising:
obtaining, based on a first time interval, the first data from the second timing to the first timing; and
in response to recognizing the state of the user as the emergency state, obtaining, based on a second time interval less than the first time interval, the first data from the first timing to the third timing.
